(19) United States
(12) Patent Application Publication
(10) Pub. No.: US 2020/0066397 A1
(43) Pub. Date: Feb. 27, 2020

(54) MULTIFACTORICAL, MACHINE-LEARNING BASED PRIORITIZATION FRAMEWORK FOR OPTIMIZING PATIENT PLACEMENT

(71) Applicant: General Electric Company, Schenectady, NY (US)

(72) Inventors: Savanoor Rai, Naperville, IL (US); Bex George Thomas, Laguna Niguel, CA (US); Andrew Day, Newtown, PA (US)

(21) Appl. No.: 16/110,703

(22) Filed: Aug. 23, 2018

(51) Int. Cl. G16H 40/20; G06Q 10/06; G06N 99/00

(52) U.S. Cl. CPC G16H 40/20; G06N 99/005; G06Q 10/067

(57) ABSTRACT

Techniques are described that employ a multifactorial, machine-learning based system and prioritization framework for optimizing patient placement to beds at a medical facility. In one embodiment, a computer-implemented is provided that comprises receiving, by a system operatively coupled to a processor, a patient placement request requesting placement of a patient to a hospital bed of the healthcare facility, wherein the request is associated with information identifying a medical service for the patient and a bed type. The method further comprises, selecting, by the system, a placement prioritization model from a set of placement prioritization models based on the medical service and the bed type, and employing, by the system, the prioritization model and state information regarding a current state of the healthcare facility to determine a prioritization score reflective of a priority level of the patient placement request.

### TECHNICAL FIELD

This application relates to computer-implemented techniques for employing a multifactorial, machine-learning based prioritization framework for optimizing patient placement to beds at a medical facility.

### BACKGROUND

Bed management is closely involved with all phases of patient stay at an inpatient medical facility. As patients with similar care requirements are admitted to the hospital from various hospital entry points, patient placements in turn will affect patient care and bed availability. Bed managers must allow best possible patient care while balancing the allocation of beds to prevent delays in care. Prioritization of patients and management of beds especially during periods of high census where there is competing demand is extremely important. Managing bed assignments is thus a critical function for patient care, reducing delays and optimizing system capacity. However, in practice, bed management is characterized by bed managers finding and firefighting crises as they occur, relying primarily on clinical knowledge, intuition, and experience.

### SUMMARY

The following presents a summary to provide a basic understanding of one or more embodiments of the invention. This summary is not intended to identify key or critical elements, or delineate any scope of the different embodiments or any scope of the claims. Its sole purpose is to present concepts in a simplified form as a prelude to the more detailed description that is presented later. In one or more embodiments described herein, systems, computer-implemented methods, apparatus and/or computer program products that employ a multifactorial, machine-learning based prioritization framework for optimizing patient placement to beds at a medical facility.

According to an embodiment, a system for prioritizing patient placements at a healthcare facility is provided that comprises a memory that stores computer executable components, and a processor that executes the computer executable components stored in the memory. The computer executable components can comprise a model generation component that identifies placement patterns and preferences of the bed managers from historical patient placement data for a medical facility. The computer executable component can also include an attribute/parameter identification/weighting component that identifies parameters and their importance by evaluating the historical patient placement data using one or more machine learning techniques to determine correlations between parameters associated with patient placement requests of similar types (e.g. same hospital service and type of care needed). This process correlates the time taken to fulfill patient placement requests with the parameters/attributes that influence the placement requests (e.g., bed occupancy levels, bed status, cleaning status, system alerts generated signifying delays in care etc.). The model generation component can further generate a placement prioritization model for the patient placement requests based on the attributes and the relative weights for the attributes, wherein the prioritization model is configured to generate prioritization scores for the patient placement requests that reflects their relative priority level to other pending placement requests of the same type.

In various embodiments, the disclosed system can also include a visualization component that facilitates generating visualizations, for rendering via a device display, that visually represent the prioritizations of the placements in a manner that allows decision makers to focus on placement decisions quickly. The system can also include a recommendation component that facilitate determining whether to wait or place the patient to a bed now, based in part on a prioritization score determined for a patient placement request using one or more of the prioritization models. Accordingly, with the disclosed system, decision makers are provided with all the relevant information to place a patient to receive the best care while reducing delays in access to care and maintain smooth patient flow

According to another embodiment, system for prioritizing patient placements at a healthcare facility is provided that comprises a memory that stores computer executable components, and a processor that executes the computer executable components stored in the memory. The computer executable components can comprise a request component that receives a patient placement request requesting placement of a patient to a hospital bed of the healthcare facility, wherein the request is associated with information identifying a medical service for the patient and a bed type. The computer executable component can further comprise a selection component that selects a placement prioritization model from a set of placement prioritization models based on the medical service and the bed type, and a scoring component that employs the prioritization model and state information regarding a current state of the healthcare facility (which includes bed status, cleaning status and unit census) to determine a prioritization score reflective of a priority level of the patient placement request.

In one or more implementations, the patient placement models can respectively comprise weighted sum models developed using machine learning analysis of historical operations data and historical patient placement data for the healthcare facility. In some implementations, the healthcare facility comprises different healthcare units which can care for patients belonging to a medical service and require a bed type. For a given medical service and bed type, machine learning analysis provides a preferred hierarchy of units ordered by most preferred to care for those patients. For every request, a prioritization score is developed for every unit in the preference hierarchy. For any given unit patients who can be placed in the unit are then ranked relative to each other based on their prioritization score. In various embodiments, the computer executable components can further comprise ranking component that determines a ranking of the patient placement request relative to other pending placement requests assigned to the first healthcare unit based on the first prioritization score and prioritization scores respectively determined for the other pending placement requests.

The computer executable components can also comprise a recommendation component that determines a recommendation regarding whether to assign the patient to a hospital bed in the first healthcare unit based on number of hospital beds that are currently available in the first hospital unit and the ranking. If placement is not possible in the most preferred unit, it can provide expected wait time based on machine learning analysis of historical wait times given similar hospital state. In addition, the system can include a visualization component that generates a visualization that visually depicts the ranking and the recommendation for rendering via a display screen of a device.

In some embodiments, elements described in the disclosed systems can be embodied in different forms such as a computer-implemented method, a computer program product, or another form.

### DESCRIPTION OF THE DRAWINGS

FIG. 1 illustrates a block diagram of an example, non-limiting system that provides a multifactorial, machine-learning based prioritization framework for optimizing patient placement in accordance with one or more embodiments of the disclosed subject matter.

FIG. 2 illustrates a block diagram of an example, non-limiting system that facilitates developing patient placement prioritization models using machine learning, in accordance with one or more embodiments of the disclosed subject matter.

FIG. 3 presents a table that defines some example bed types categorized by level of care categories that can be used to distinguish between different types or clusters of bed requests in accordance with one or more embodiments of the disclosed subject matter.

FIG. 4 presents a table identifying example attributes that may or have been determined to influence when and how patient placement requests are fulfilled in accordance with various embodiments described herein.

FIG. 5 presents a table describing some example importance scores determined for attributes associated with determining a priority level of one example type of patient placement request in accordance with one or more embodiments of the disclosed subject matter.

FIG. 6 presents a chart demonstrating example weights of different parameters reflective of their relative importance in association with determining priority levels of several types of patient placement requests in accordance with one or more embodiments of the disclosed subject matter.

FIG. 7 presents a chart demonstrating example weights of different parameters reflective of their relative importance in association with determining priority levels of patient placement requests in the critical care unit, in accordance with one or more embodiments of the disclosed subject matter.

FIG. 8 presents a chart demonstrating example weights of different parameters reflective of their relative importance in association with determining priority levels of patient placement requests in the floor unit, in accordance with one or more embodiments of the disclosed subject matter.

FIG. 9 presents a chart comparing weighting of factors amongst the critical care and floor unit clusters for medicine, neurology and surgery service line, in accordance with one or more embodiments of the disclosed subject matter.

FIG. 10 illustrates a block diagram of an example, non-limiting system that provides for employing a multifactorial, machine-learning based prioritization framework for optimizing patient placement in real-time, in accordance with one or more embodiments of the disclosed subject matter.

FIG. 11 illustrates a block diagram of another example, non-limiting system that provides for employing a multifactorial, machine-learning based prioritization framework for optimizing patient placement in real-time, in accordance with one or more embodiments of the disclosed subject matter.

FIG. 12 illustrates a block diagram of another example, non-limiting system that provides for employing a multifactorial, machine-learning based prioritization framework for optimizing patient placement in real-time, in accordance with one or more embodiments of the disclosed subject matter.

FIG. 13 presents an example graphical user interface (GUI) including an aggregate cluster view visualization of patient placement requests in accordance with one or more embodiments described herein.

FIG. 14 presents an example GUI including a detailed cluster view visualization that provides a detailed view of the bed requests for acute beds types in the medicine floor service line, in accordance with one or more embodiments described herein.

FIG. 15 presents an example GUI including another detailed cluster view visualization that provides a detailed view of the bed requests within a specific medical service line in accordance with one or more embodiments described herein.

FIG. 16 presents an example GUI including another detailed cluster view visualization that provides a detailed view of the bed requests for acute beds types in the medicine floor service line, in accordance with one or more embodiments described herein.

FIG. 17 presents an example GUI including a unit view visualization in accordance with one or more embodiments described herein.

FIG. 18 presents an example GUI including another unit view visualization with a pop-up display window in accordance with one or more embodiments described herein.

FIGS. 19 and 20 respectively present another detailed cluster view visualization and another detailed unit view visualization that can be presented to a bed manager in association an example use-case of the subject patient placement prioritization application, in accordance with one or more embodiments of the disclosed subject matter.

FIG. 21 presents a configuration GUI that allows the bed managers to adjust the weighting of several factors included in a prioritization model to reflect their operational policies in accordance with one or more embodiments of the disclosed subject matter.

FIG. 22 presents another configuration GUI that allows the bed managers to change their preference hierarchy to optimize care for patients in accordance with one or more embodiments of the disclosed subject matter.

FIG. 23 presents an example search GUI that can be used to find specific patient placement requests in accordance with one or more embodiments described herein.

FIG. 24 illustrates a block diagram of an example, non-limiting system that provides for employing a multifactorial, machine-learning based prioritization framework for optimizing patient placement in real-time, in accordance with one or more embodiments of the disclosed subject matter.

FIG. 25 illustrates an example, high level flow diagram of a computer-implemented process for employing a multifactorial, machine-learning based prioritization framework to facilitate optimizing patient placement, in accordance with one or more embodiments of the disclosed subject matter.

FIG. 26 illustrates another example, high level flow diagram of a computer-implemented process for employing a multifactorial, machine-learning based prioritization framework to facilitate optimizing patient placement, in accordance with one or more embodiments of the disclosed subject matter.

FIG. 27 illustrates another example, high level flow diagram of a computer-implemented process for employing a multifactorial, machine-learning based prioritization framework to facilitate optimizing patient placement, in accordance with one or more embodiments of the disclosed subject matter.

FIG. 28 illustrates a block diagram of an example, non-limiting operating environment in which one or more embodiments described herein can be facilitated.

### DETAILED DESCRIPTION

The following detailed description is merely illustrative and is not intended to limit embodiments and/or application or uses of embodiments. Furthermore, there is no intention to be bound by any expressed or implied information presented in the preceding Summary section or in the Detailed Description section.

Efficient patient placement at an inpatient medical facility enables patients to have quick access to appropriate medical care and helps the medical facility manage capacity by eliminating bottlenecks. In many hospitals, patients with bed requests are pre-assigned a medical unit by the bed management team and subsequently assigned a bed by the pre-assigned unit. In theory bed management is a simple task (a bed is assigned to patient when it is available). But bed management has a significant impact on patient care, patient flow, and hospital capacity. To optimize patient care and capacity, bed placements need to be carefully considered and executed. Bed managers, typically use a set of assignment rules and patient care requirements to decide where the patient should go. Some tend to look at clinical factors and are pressed for time to look at multiple other factors that affect capacity and patient flow (upstream or downstream effects). Prioritizing patient placements is a challenge with bed managers and to truly achieve throughput improvement, it will require organizational alignment. This in turn requires placement prioritization and subsequent decision making to have a transparent common framework, especially during periods of high census when there is competing demand for beds. When census levels are high in a hospital, the stakes are high and it becomes important to properly structure the problem of placement prioritization and explicitly evaluate multiple factors. In the decision to prioritize placements, there are not only complex issues involving multiple factors but there are also multiple patients deeply affected by the consequences. Hence structuring the placement prioritization problem well and considering multiple factors leads to better patient care and reduced delays in care.

The subject disclosure is directed to systems, computer-implemented methods, apparatus and/or computer program products that provide techniques for improving patient care and reduce delays in care using a systematic, machine learning based approach to prioritize the placement of patients. In this regard, the disclosed computer-implemented techniques facilitate determining how to prioritize patient placements in real-time to enable optimal patient care with minimum delays, reduce congestion in key medical facility units, and maintain smooth patient flow.

In one or more embodiments, the disclosed techniques can involve a model development process and a model application process. The model development process can involve using machine learning analysis of historical patient placement, hospital states such as occupancy levels, bed occupancy and cleaning status, workflow data and to develop one or more prioritization models that provide prioritization information regarding how to prioritize patient placements in a dynamic inpatient medical environment based on various clinical and operational factors. The model development process can be an offline process and computationally very efficient. The model application process can involve applying the one or more prioritization models in a live clinical setting to generate recommendation information regarding how to prioritize patient placements. The model application process is thus an online or live process that can provide real-time feedback in current clinical scenarios.

In various embodiments, the model development process can involve using a multifactorial or multicriteria decision making (MDC), machine-learning based approach to develop one or more patient placement prioritization models based on analysis of historical information regarding patient placements, patient flow, patient quality of care, facility workflow and the like. By using an MDC machine learning approach, the disclosed techniques can identify and evaluate relationships between many complex and interrelated factors that impact patient care and patient flow, enabling an enterprise based view of patient placement prioritization problem. Using an MDC technique, it is possible to incorporate user provided parameters not available from historical analysis and weight them subjectively. In particular, with the disclosed techniques, a machine learning based model development process is provided. This model development process uses historical data integrated from multiple sources to quantitatively and qualitatively evaluate of a large number of interrelated clinical and operational factors to learn how these factors effect prioritization of patient placements. For example, historical bed management data, hospital operations data, and the like, can be evaluated using machine learning techniques to learn placement patterns and estimate the importance (weights) of several factors that influence placements. Based on the machine learning evaluation, one or more prioritization models can be developed that correlate the manner in which different clinical and operational factors impact an optimal prioritization scheme for a plurality of bed requests. In this regard, the optimal prioritization scheme can be based on fulfilling and/or balancing one or more defined goals, including but not limited to: ensuring quality patient care in line with defined medical care standards, minimizing patient placement time delays, reducing congestion in medical units, and maintaining smooth patient flow. In some embodiments, the optimal patient placement prioritization scheme can also balance fulfilling patient preferences, medical practitioner preferences, minimizing financial loss, and/or maximizing financial gain.

In some embodiments, the disclosed techniques can determine an optimal prioritization scheme for prioritizing patient placement requests by formulating the task as a prioritization problem that characterizes the optimal prioritization scheme for prioritizing patient placement requests in the form of a prioritization score for each patient placement request. With these embodiments, the prioritization models can generate prioritization scores for patient placement requests that represent relative priority levels recommended for fulfilling the respective requests to achieve one or more of the goals noted above. The prioritization models can define weighted values for and relationships between, one or more clinical and operational factors that impact prioritization scores for bed placement requests. The weighted values and/or relationships can be determined based on machine learning analysis of the historical data. In this regard, a prioritization score enables ranking of a placement request by accounting for various clinical and operational factors associated with the request in view of the one or more defined goals noted above. For example, a high prioritization score associated with a particular patient placement request (relative to other requests with the same medical service and bed type) can indicate that the request should be fulfilled prior to another request for the same bed that is associated with a lower prioritization score in order to achieve optimal patient care and reduced delays.

In one or more embodiments, learning and model development can be carried out for different distinct types of bed requests that are respectively characterized by a combination of a specific hospital service and bed type. With these embodiments, a different prioritization model can be developed for each (or in some implementations one or more) distinct combination of hospital service and bed type. In some embodiments, different prioritization models can also be developed for each (or in some implementations one or more) medical unit at the healthcare facility. With these embodiments, leaning and model development can be carried out for each distinct combination of hospital service and bed type within each specific medical unit. In this regard, each medical unit can be associated with a plurality of different prioritization models that are tailored to that medical unit, wherein each periodization model within the medical unit is configured to a distinct combination of medical service and bed type. For example, a patient is often a candidate for medically appropriate placement in several different units. When determining how to prioritize placing the patient, the disclosed system can be configured to run the distinct (unit specific) machine learning based scoring prioritization model/algorithm for each medical unit where the patient can be placed. As a result, a different prioritization score can be generated for the request for each of the different medical units. According, based on the patient's prioritization scores for the respective medical units, the patient may achieve recommended potential placement in more than one unit at a time. In this case, if one of the optional medical units is known to be a preferred unit, the preferred unit may be selected if it is one in which the patient was ranked for potential placement. However, in other implementation, the system may recommend, (and/or an operator may also choose), to place the patient in a less preferred unit in situations where the less preferred unit is less highly in demand. For example, this allows for the unit with a long list of patients needing placement to be used for patients for whom this more available unit is not an option.

The developed prioritization models can be stored in a machine learning database and employed to facilitate determining how to prioritize patient placements in real-time in an actual inpatient environment. In this regard, the model application process can use the prioritization models and real-time state information regarding the current state of a medical facility to provide recommendations regarding how to prioritize received or currently pending bed requests. In one or more embodiments, the model application process can involve using the appropriate prioritizations model to determine prioritization scores for each (or in some implementations one or more) unfulfilled bed request. A linear additive utility function can further be employed to rank the placement requests and determine which placement requests should be fulfilled based on their respective rankings and current bed availability. In some implementations, the model application process can also employ artificial intelligence and predictive analytics to determine when and/or how to assign patients to beds based on predicted bed availability, predicted congestion levels, distribution of various types of predicted placement requests to be received within an upcoming time window, and the like. Information regarding patient placement prioritization scores, and associated recommendations regarding when, where and how to place patients in a live inpatient setting can further be provided to clinicians/bed managers in real-time in a structured and meaningful way to facilitate efficient bed management.

In various embodiment, requests for placing a patient to a bed at a medical facility are referred to herein as patient placement requests or simply placement requests. In this regard, the term bed is used to generally refer to a bed where a patient receives a medical care at an inpatient medical facility (e.g., a hospital, a clinic, a medical specialty unit, a nursing home, an assisted living facility, etc.). However, the term bed as used herein can encompass any type of designated physical location where a limited number of patients, (e.g., a single patient in most implementations), can be placed to receive a specific type of medical care. For example, the term bed can encompass mobile beds, chairs, tables, rooms, etc.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

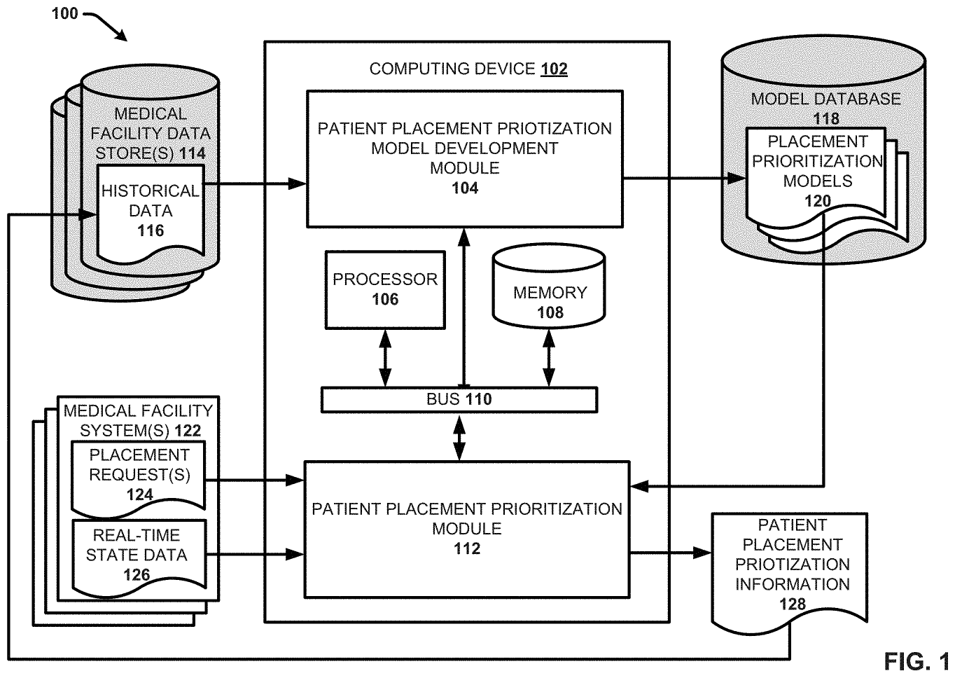

Turning now to the drawings, FIG. 1 illustrates a block diagram of an example, non-limiting system 100 that provides a multifactorial, machine-learning based prioritization framework for optimizing patient placement in accordance with one or more embodiments of the disclosed subject matter. Embodiments of systems described herein can include one or more machine-executable components embodied within one or more machines (e.g., embodied in one or more computer-readable storage media associated with one or more machines). Such components, when executed by the one or more machines (e.g., processors, computers, computing devices, virtual machines, etc.) can cause the one or more machines to perform the operations described. For example, in the embodiment shown, system 100 includes a computing device 102 that includes and a patient placement prioritization model development module 104 and a patient placement prioritization module 112, which can respectively correspond to machine-executable components. System 100 also includes various electronic data sources and data structures comprising information that can be read by, used by and/or generated by the patient placement prioritization model development module 104 and/or the patient placement prioritization module 112. For example, these data sources and/or data structures can include but are not limited to: medical facility data stores 114, historical data 116, model database 118, placement prioritization models 120, medical facility systems 122, real-time state data 126, placement requests 124 and patient placement prioritization information 128.

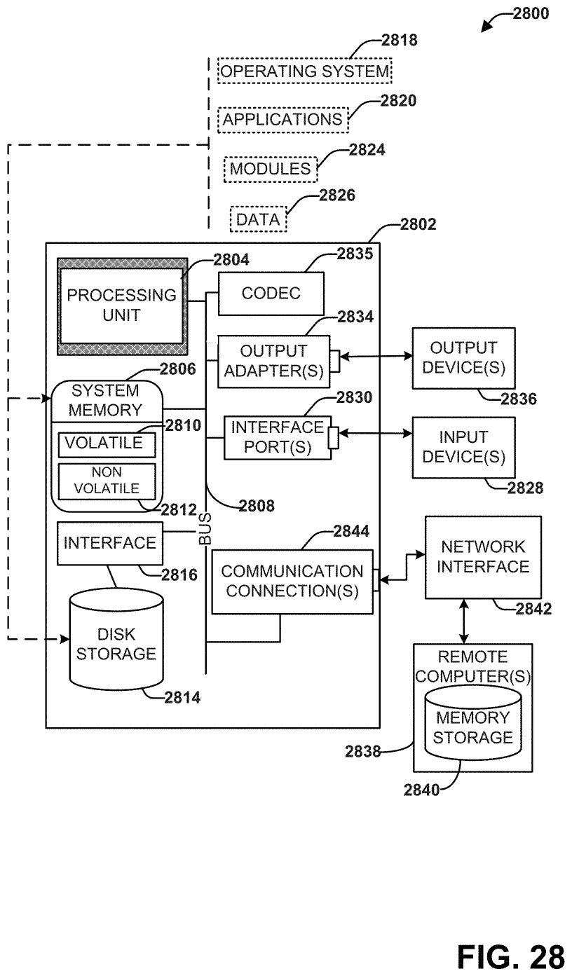

The computing device 102 can include or be operatively coupled to at least one memory 108 and at least one processor 106. The at least one memory 108 can further store executable instructions (e.g., the patient placement prioritization model development module 104 and the patient placement prioritization module 112) that when executed by the at least one processor 106, facilitate performance of operations defined by the executable instruction. In some embodiments, the memory 108 can also store one or more of the various data sources and/or structures of system 100 (e.g., one or more of the medical facility data stores 114, the historical data 116, the model database 118, the placement prioritization models 120, the medical facility systems 122, the patient placement prioritization information 128 and the like). In other embodiments, one or more of the various data sources and structures of system 100 can be stored in other memory (e.g., at a remote device or system), that is accessible to the computing device 102 (e.g., via one or more networks). The computing device 102 can further include a device bus 110 that communicatively couples the various components and data sources of the computing device 102 (e.g., the patient placement prioritization model development module 104 and the patient placement prioritization module 112, the processor 106, the memory 108). Examples of said processor 106 and memory 108, as well as other suitable computer or computing-based elements, can be found with reference to FIG. 28, and can be used in connection with implementing one or more of the systems or components shown and described in connection with FIG. 1 or other figures disclosed herein.

In some implementations, the computing device 102, and/or the various components and data sources of system 100 (and other systems described herein) can be communicatively connected via one or more networks. Such networks can include wired and wireless networks, including but not limited to, a cellular network, a wide area network (WAD, e.g., the Internet) or a local area network (LAN). For example, the computing device 102 can communicate with one or more medical facility data stores 114, one or more medical facility systems 122, and the like, (and vice versa), using virtually any desired wired or wireless technology, including but not limited to: wireless fidelity (Wi-Fi), global system for mobile communications (GSM), universal mobile telecommunications system (UMTS), worldwide interoperability for microwave access (WiMAX), enhanced general packet radio service (enhanced GPRS), third generation partnership project (3GPP) long term evolution (LTE), third generation partnership project 2 (3GPP2) ultra mobile broadband (UMB), high speed packet access (HSPA), Zigbee and other 802.XX wireless technologies and/or legacy telecommunication technologies, BLUETOOTH®, Session Initiation Protocol (SIP), ZIGBEE®, RF4CE protocol, WirelessHART protocol, 6LoWPAN (IPv6 over Low power Wireless Area Networks), Z-Wave, an ANT, an ultra-wideband (UWB) standard protocol, and/or other proprietary and non-proprietary communication protocols. The computing device 102 can thus include hardware (e.g., a central processing unit (CPU), a transceiver, a decoder), software (e.g., a set of threads, a set of processes, software in execution) or a combination of hardware and software that facilitates communicating information between the computing device 102 and externals systems, sources and devices.

In various embodiments, the patient placement prioritization model development module 104 can perform various functions that are directed to developing one or more placement prioritization models 120. The one or more placement prioritization models 120 can thereafter be used by the patient placement prioritization module to generate patient placement prioritization information 128 regarding how to prioritize received placement requests 124 in an actual or live setting to optimize patient flow and patient care.

A good patient placement process ensures patients are placed in appropriate medical care units at appropriate times to receive necessary care and eliminates delays in transferring patients to beds in the appropriate care units. However, patient placement decisions are often complex and involves multiple criteria. For example, in a dynamic hospital setting patients are treated, admitted, discharged or transferred throughout various different specialty areas and medical units. Given a fixed number of beds, inevitably scenarios arise wherein certain bed requests cannot be fulfilled immediately (e.g., without delay) and/or in the preferred manner as requested. Determining how to adapt and prioritize placing patient placement requests in high volume and dynamic inpatient settings should therefore strive to minimize delays associated with fulfilling requests and minimize congestion/bottlenecks, while facilitating provision of optimal patient care.

Most bed managers generally prioritize patients competing for a same bed based on urgency of patient care needs. However, determining when and where to assign a particular patient to a hospital bed to achieve optimal patient care for all patients with minimum delays in access to care throughout the healthcare facility requires consideration of many additional interrelated and multidimensional clinical and operational factors. For example, these additional factors can include defined operational policies of the healthcare facility that dictate requirements regarding the type of bed a patient needs or can be placed, what medical unit is appropriate for providing the medical service needed by the patient, specific clinicians that are authorized and available to treat the patients, and the like. These additional factors can also include for example, future needs and complications associated with treating the patient as well as the current needs and workflows associated with other patients at the healthcare facility. In another example, factors such as the current location of a patient requesting a bed, transfers times and distances associated with placing the patient to certain beds, location and distribution of needed medical supplies needed for treating the patient, current occupancy levels, Environmental Services or housekeeping/cleaning (EVS) status (e.g., which reflects a state of the bed between patients, including cleanliness, sheets changed, instruments replaced, etc.), predicted future occupancy levels, current bed availably, predicted future bed availability and the like, can all impact patient flow and quality of care, and should thus be considered when determining when and where to place a patient. Complex problems like these therefore require transparency of policies, breakdown of the complexity into manageable components and a way to bring all the components together for systematic and robust decision making.

The patient placement prioritization model development module 104 can employ one or more machine learning techniques to learn, based on historical healthcare facility operational and performance data, how various interrelated and multidimensional clinical and operational factors associated with patient placements requests influence how to prioritize the patient placements to achieve optimal patient flow and patient care to all patients throughout the healthcare facility. For example, in one or more implementations, the patient placement prioritization model development module 104 can employ one or more machine learning techniques to learn placement patterns based on historical data regarding bed management decisions, patient flows and the like. In some implementations, the patient placement prioritization model development module 104 can further learn what clinical, operational and/or contextual factors influence patient placements. The patient placement prioritization model development module 104 can further learn correlations between how certain factors influence how to prioritize patient placement requests in order to achieve optimal patient flow and care (e.g., based on historical performance data regarding patient flows, clinical and financial outcomes associated with patient placement decisions and the like)

In the embodiment shown, the historical medical facility operational and performance data is represented as historical data 116 and is provided by one or more medical facility data stores 114. For example, the one or more medical facility data stores 114 can include one or more internal databases of a medical organization or facility that system 100 is applied to in association with tailoring patient placement prioritization schemes to optimize one or more goals (e.g., improved patient flow and care) of the healthcare facility. It should be appreciated however that the source and location of the historical data 116 can vary. In various embodiments, the historical data 116 can be collated from various disparate data sources associated with the medical facility (e.g., bed management systems, patient record systems, scheduling systems, billing systems, administration systems, performance evaluation systems, etc.).

The historical data 116 can thus include several types of historical medical facility operational and performance data regarding bed management, patient placements, patient flows, clinical and financial outcomes associated with patient placement decisions, facility operational data and the like. For example, the historical medical facility operation and performance data can include bed management information, including information regarding timing and distribution of received patient placement requests, timing of fulfilment of the patient placement requests, order of fulfillment of the patient placement requests, how the patient placement requests are filled (e.g., what bed and medical unit the patient is place in response to the request), data regarding when and how available beds are assigned, timing of bed availability, distribution of bed availability, EVS status, delays associated with be cleaning and preparation, and the like and the like. The historical medical facility information can also include patient records from various medical units and specialties throughout a healthcare facility. Such patient records can include for example, patients treated, clinicians involved in the patient's treatment, workflows of patients treated (e.g., from intake to discharge), diagnosis, procedures performed, complications that arose, and the like. The historical medical facility information can also include clinical and financial outcomes information such as information (which can be correlated to patient flows and bed assignment). In another example, the historical medical facility information can include scheduling information for patients (e.g., regarding medical appointments at locations throughout the healthcare facility), scheduling information for clinicians, and the like.

By evaluating such historical data 116 as described above, the patient placement prioritization model development module 104 can learn how to prioritize patient placement based on a plurality of complex and interrelated factors that can affect patient flow and patient quality of care, (and in some implementations other financial performance of the medical facility). The patient placement prioritization model development module 104 can further develop one or more placement prioritization models 120 that can facilitate automatically determining how to prioritize placing patients to achieve optimal flow and care based on these many complex and interrelated factors. For example, the one or more placement prioritization models 120 can define relationships between, and relative importance weights of, various factors associated with different patient placement requests that have an impact on patient flow and optimal patient care as a function of patient placement priority. In this regard, the placement prioritization models 120 can relate multiple criteria associated with respective patient placement requests and contexts associated with the respective patient placement requests to a prioritization scheme for fulfilling the requests to achieve optimal patient flow and provide optimal patient care. In this regard, optimal patient flow can be characterized by at least minimizing delays between fulfilling bed requests and reducing congestion/bottlenecks in medical providing units. Optimal patient care can be characterized by timely delivering the necessary and most appropriate care to all patients in accordance with defined medical standards of care, while minimizing errors and complications. In some embodiments, the one or more placement prioritization models 120 can also be configured to determine a priority scheme for fulfilling patient placement requests as a function of minimizing financial loss and/or maximizing financial gain of the healthcare facility.

The one or more placement prioritization models 120 developed by the patient placement prioritization model development module 104 can be stored in a model database 118 where they can be accessed by the patient placement prioritization module for application in real-time operation of the healthcare facility. In the embodiment shown, the model database 118 is located external to the computing device 102. However, it should be appreciated that model database 118 can be located in memory 108, or another suitable network accessible device/system.

The patient placement prioritization module 112 can be configured to employ the one or more placement prioritization models 120 in association with real-time state data 126 regarding a current operational state of an inpatient healthcare facility, to facilitate determining how to prioritize the placement requests 124 that are received or otherwise currently pending at the healthcare facility. In this regard, patient placement prioritization module 112 can be communicatively coupled to one or more electronic data systems of a medical facility, identified in system 100 as medical facility systems 122. The medical facility systems 122 can include one or more computer systems that can provide information regarding received or pending placement requests 124 and real-time state data 126 regarding a current operational state of the healthcare facility. These medical facility systems 122 can include different systems designed to manage, monitor, or otherwise facilitate various operations of the healthcare facility, and can include internal systems, open source systems, onsite systems, remote systems, cloud based systems and the like. In this regard, the patient placement prioritization module 112 can aggregate useful and relevant information from various disparate sources to facilitate determining how to prioritize patient placement schemes.

For example, in various embodiments, information regarding placement requests 124 received at the healthcare facility can be provided by or otherwise extracted from one or more bed management systems of the healthcare facility that receive and manage information regarding placement requests 124 at the healthcare facility. For instance, in some implementations, the bed management systems can include a system that aggregates and manages all bed requests for the healthcare facility, including bed requests of different types and/or bed requests associated with different medical units throughout the healthcare facility. In another implementation in which different medical units of the healthcare facility employ separate bed management systems, the patient placement prioritization module 112 can access the respective bed management systems separately. In this regard, the architecture of the bed management system or systems employed by the healthcare facility for managing placement requests can vary. In association with managing placement requests, the bed management system(s) can provide a variety of information associated with the patient placement requests. For example, the placement requests 124 can be associated with information regarding details of the requests, such as but not limited to: the type of bed and medical service associated with the request, one or more preferred destination medical units for fulfilling the request, an order type associated with the request, priority information defined for the request, assignment urgency information associated with the request, a type of room requested, event information regarding what triggered the request, clinician instructions regarding specific care instructions for the patient, and the like. The bed management systems can also provide information identifying timing of reception of respective placement requests 124, current status of the respective requests (e.g., pending, fulfilled, on-hold, etc.), total number of pending requests at the healthcare facility, total number of pending requests per unit, number of pending requests clustered by request type, and the like.

The placement requests 124 can also include information regarding the patient for which a request is made, such as but not limited to: information identifying the patient, a current state/condition of the patient, demographic information for the patient, information regarding preferences of the patient, insurance information for the patient, and the like. In some implementations, the medical facility systems 122 can include an electronic health record (EHR) system that provides historical health/medical information for respective patients being treated at the healthcare facility, including records for patients associated with placement requests. The placement requests 124 can also be associated with information identifying the current location of the patient, a categorization of the patient's current location, information regarding one or more policies required for the requests and the like.

In addition to bed management systems that provide information regarding placement requests 124 received at the healthcare facility, the medical facility systems 122 can further include various data sources/systems that can provide information regarding a current state of the healthcare facility (e.g., real-time state data 126), that may be relevant in association with determining when and where to place patients in association with fulfilling the placement requests 124. For example, the real-time state data 126 can include at least occupancy data regarding the current occupancy levels of beds throughout the medical facility. In this regard, the occupancy data can not only identify the number of patients currently admitted at the inpatient healthcare facility, but also current status of all (or in some implementations, one or more) beds at the medical facility, such as available, unavailable, in-repair, requiring cleaning, being cleaned, etc. The occupancy data can also be categorized by medical unit (e.g., number of beds available in the surgery floor unit). The occupancy data can also include timeline information regarding the duration of time the respective beds are associated with a particular status (e.g., occupied by patient John Doe since 11 am).

In some implementations, the occupancy data can also include forecasted information regarding predicted availability of beds (e.g. anticipated timing when an occupied bed will become available). For example, in implementations in which known procedures and/or courses of care are associated with known durations or points in time or timelines when a patient will be released, transferred or the like, information regarding the timing of availability of certain beds that are currently in use can be predicted with relatively high accuracy. In this regard, the medical facility systems 122 can include a monitoring system that monitors the status of patients and/or courses of patient care and predicts information regarding when beds will become available. In other implementations in which some medical services provided by the medical facility involve prescheduled appointments where patient occupy beds at the medical facility, medical facility systems 122 can include an appointment scheduling system that including information identifying scheduled appointments at the medical facility. With these embodiments, the appointment scheduling information can be used to forecast when (and how long) certain beds at the medical facility will be occupied for prescheduled appointments.

In some implementations, the real-time state data 126 can also include information provided by one or more medical supply systems regarding the current location, availability and status of medical supplies throughout the healthcare facility. In this regard, patients for which treatment requires certain medical supplies can be assigned to beds where the medical supplies are available. In another example, if a certain medical supply or equipment required for treatment of a first patient is unavailable (e.g., an implantable device, an organ for transplant, etc.), a second patient who arrived after the first patient that does not require the certain medical supply can be assigned to the bed in the meantime. In some implementations, the medical supplies can include mobile beds where patients can be placed. In accordance with these implementations, the real-time state data 126 can also include information regarding current locations and status of mobile beds. The real-time state data 126 can also include information regarding the current activity and locations of medical personal and clinicians throughout the healthcare facility. In this regard, patients requiring certain clinicians and/or a certain number of clinicians can be assigned to the appropriate beds where the clinicians are available at the appropriate times when the clinicians are available. In some implementations, information regarding current availability of clinicians and/or upcoming availability of clinicians can be provided and/or determined from scheduling information for the clinicians regarding their known work schedules, appointment schedules, and the like. The real-time state data 126 can also include alert or notification data provided by a notification system regarding possible complications, high priority events, emergency events (e.g., associated with one or more current patients, associated with a catastrophic event in the area near the medical facility involving an influx of a mass number of patients), interruptions or changes to normal operations, faulty equipment, and the like.

Accordingly, the patient placement prioritization module 112 can be configured to monitor the medical facility systems 122, to identify information regarding newly received and/or pending placement requests 124 and the associated real-time state data 126 that can have an impact on when and where placement requests 124 are fulfilled. In one or more embodiments, when a new placement request 124 is received, the patient placement prioritization module 112 can be configured to identify and select an appropriate model of the placement prioritization models 120 for the patient placement request included in the model database 118 based on one or more attributes associated with the request. For example, in some embodiments, the placement prioritization models 120 can include different models tailored to different types of request, wherein each type of request is based on a different combination of bed type, medical service, and/or medical unit associated with the request. In this regard, the type of the request can define or otherwise indicate what type of bed and/or medical unit at the healthcare facility where the patient can be placed.

The patient placement prioritization module 112 can further employ the placement prioritization model 120 to determine the patient placement prioritization information 128 regarding how to prioritize fulfilling the request relative to other pending requests for the same type of bed in the same medical unit. The patient placement prioritization information 128 can thus provide informed insight regarding how to optimize patient placement in a real-time hospital setting, considering the multiple factors associated with the requests and dynamic state of the medical facility. The patient placement prioritization module 112 can further provide the patient placement prioritization information 128 to bed managers via a suitable electronic notification or reporting mechanism to facilitate guiding the bed mangers in association with deciding how and when to assign patients to appropriate beds in real-time. Because the placement prioritization models 120 are configured to determine a prioritization scheme for fulfilling the request as a function of achieving optimal patient flow, optimal patient quality of care, optimal medical facility performance (e.g., in terms of clinical and financial performance), and the like, the patient placement prioritization information 128 can reflect a priority scheme for fulfilling the placement requests 124 that will likely achieve these goals. In this regard, the placement prioritization scheme or schemes reflected in the patient placement prioritization information 128 can directed to optimize placement efficiency and patient flow (e.g., no empty beds waiting to be filled, reduced lag time between reception of a request and assignment of the request to a bed), improve clinical outcomes, minimize clinical errors, improve financial outcomes, improve patient quality of care, and the like.

For example, in one or more embodiments, the patient placement prioritization module 112 can select an appropriate prioritization model from the placement prioritization models 120 from the model database 118 for a newly received placement request 124 based on request details associated with the placement request (e.g., bed type, medical service, etc.). The patient placement prioritization module 112 can further use the request details along with other attributes associated with request (e.g., current location of the patient, order type, assignment urgency, patient class, policy of the destination unit, etc.) and real-time state data 126 regarding a current context of the healthcare facility (e.g., occupancy levels of one or more suitable destination medical units where the patient can be placed, bed availability, clinician availability, EVS status, etc.) as input to the selected placement prioritization model to determine a priority level or priority score of the request. Using the same placement prioritization model, the patient placement prioritization module 112 can further determine relative priority levels for other pending placement requests competing for the same bed/medical unit. The patient placement prioritization module 112 can further generate patient placement prioritization information 128 (e.g., as output data) identifying the priority level or score of the new patient placement request and the priority levels or scores of any other pending patient placement requests competing for the same type of bed in the same unit. In some implementations, the patient placement prioritization module 112 can further rank all requests competing for the same bed/unit based on their priority levels or scores. The patient placement prioritization module 112 can further recommend bed assignments based on bed availably and relative ranking of the respective requests. For example, scores can be sorted from highest to lowest and ranked in each unit. Based on the availability of beds, the requests with ranks less than or equal to the available beds in that unit can be prioritized.

In some embodiments, the patient placement prioritization information 128 can be added to the historical data 116 and used by the patient placement prioritization model development module 104 to facilitate updating and tailoring the placement prioritization models 120. In this regard, patient placement prioritization schemes determined using the placement prioritization models 120 can be compared to actual prioritization schemes applied by the healthcare facility, (e.g., including actual patient placement schemes that followed the recommended patient placement schemes represented in the patient placement prioritization information 128 and alternative schemes that did not follow the patient placement prioritization information 128), to learn if and how the placement prioritization models 120 can be adapted to further optimize achieving/balancing of the defined goals of the respective placement prioritization models. As described above, these goals can include but are not limited to: better patient flow characterized be less delay time in fulfilling requests, less bottlenecks, etc., better patient quality of care characterized by provision of necessary care to a level of highest quality and patient satisfaction, shorter length of stay (LOS), fulfilment of patient preferences, et., and improved clinical and financial outcomes, etc.). For example, the patient placement prioritization model development module 104 can determine whether the recommended patient placement prioritization schemes were actually implemented and, if so, when they worked or did not work to achieve one or more of the goals above). Based on this analysis, the patient placement prioritization model development module 104 can further adjust and optimize the placement prioritization models 120 to better achieved the goals defined above.

Many of the disclosed embodiments of the patient placement prioritization model development module 104 and/or the patient placement prioritization module 112 described herein can employ machine learning analysis of the historical data 116 and/or the information provided by the one or more medical facility systems 122 to make determinations and/or inferences regarding development of the placement prioritization models and/or applying the patient placement prioritization models to determine how to prioritize the placement requests 124 received in a live inpatient environment. In this regard, the determinations and/or inferences can be based on entirety or a subset of the historical data 116 included in the medical facility data stores 114 and the medical facility systems, and can provide for reasoning about or inferring states of the system (e.g., system 100 and the like), environment, etc. from a set of observations as captured via events and/or data. For example, an inference can be employed to identify a specific context or action, or can generate a probability distribution over states, for example. The inference can be probabilistic (e.g., the computation of a probability distribution over states of interest can be based on a consideration of data and events). An inference can also refer to techniques employed for composing higher-level events from a set of events and/or data. Such an inference can result in the construction of new events or actions from a set of observed events and/or stored event data, whether or not the events are correlated in close temporal proximity, and whether the events and data come from one or several event and data sources. Various classification (explicitly and/or implicitly trained) schemes and/or systems (e.g., support vector machines, neural networks, expert systems, Bayesian belief networks, fuzzy logic, data fusion engines, etc.) can be employed in connection with performing automatic and/or inferred action in connection with the claimed subject matter.

A classifier can map an input attribute vector, x=(x1, x2, x4, x4, xn), to a confidence that the input belongs to a class, such as by f(x)=confidence(class). Such classification can employ a probabilistic and/or statistical-based analysis (e.g., factoring into the analysis utilities and costs) to prognose or infer an action that a user desires to be automatically performed. A support vector machine (SVM) is an example of a classifier that can be employed. The SVM operates by finding a hyper-surface in the space of possible inputs, where the hyper-surface attempts to split the triggering criteria from the non-triggering events. Intuitively, this makes the classification correct for testing data that is near, but not identical to training data. Other directed and undirected model classification approaches include, e.g., naïve Bayes, Bayesian networks, decision trees, neural networks, fuzzy logic models, and probabilistic classification models providing different patterns of independence can be employed. Classification as used herein also is inclusive of statistical regression that is utilized to develop models of priority.

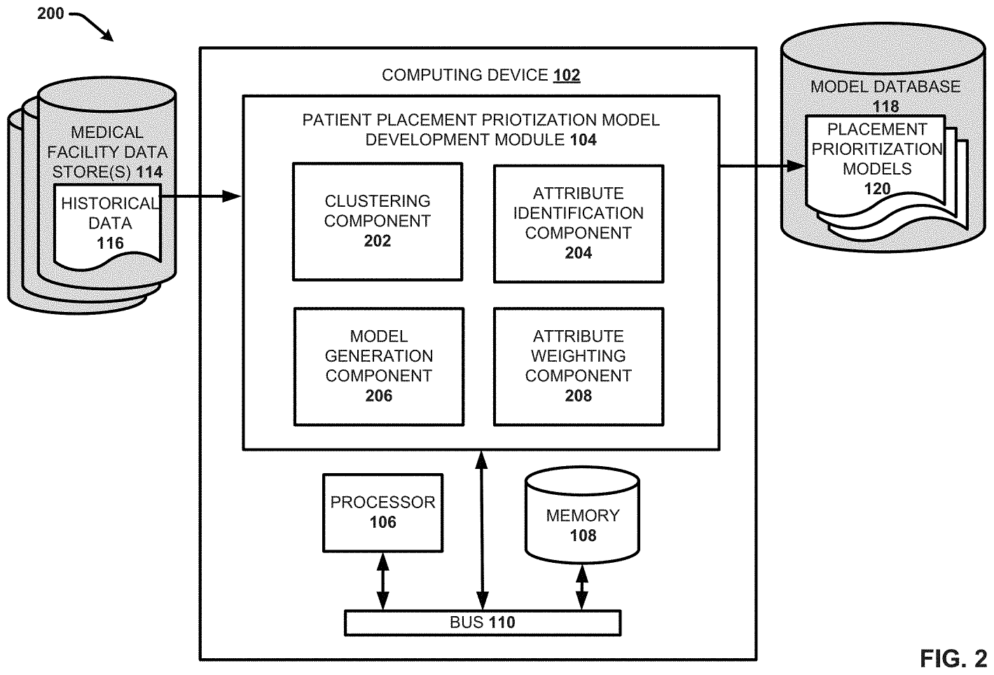

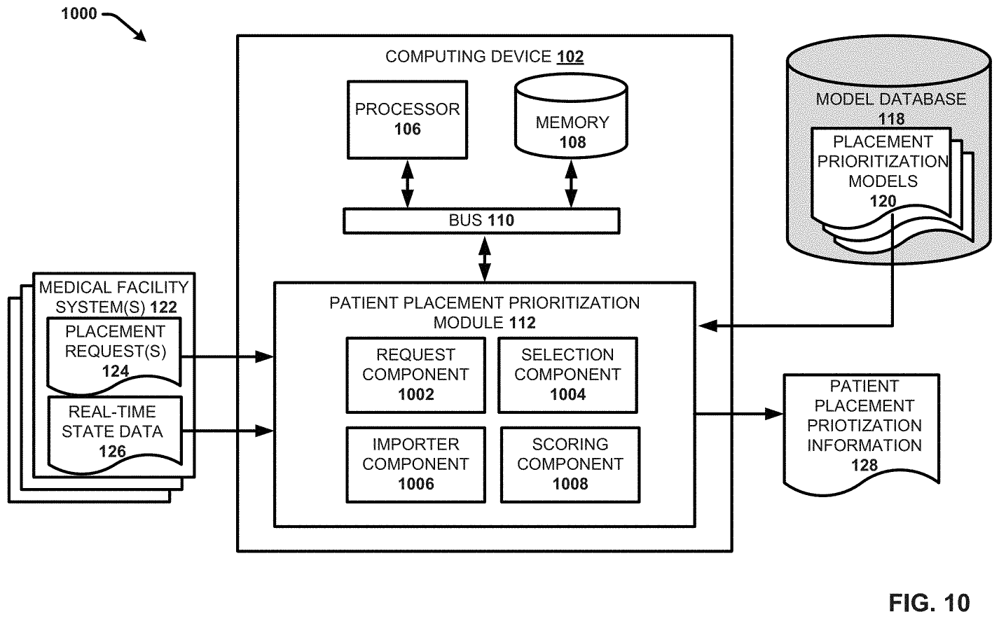

Additional details regarding the features and functionalities of the patient placement prioritization model development module 104 are described with reference to FIGS. 2-9. Additional details regarding the features and functionalities of the patient placement prioritization module 112 are described with reference to FIGS. 10-24.

With reference now to FIG. 2, presented is an example, non-limiting system 200 that facilitates developing one or more patient placement prioritization models (e.g., placement prioritization models 120) using machine learning, in accordance with one or more embodiments of the disclosed subject matter. In this regard, system 200 presents some additional example components of the patient placement prioritization model development module 104 that can perform different operations associated with developing the one or more placement prioritization models 120. In one or more embodiments, system 200 is subsystem of system 100 (e.g., system 100 can include system 200, and vice versa). Repetitive description of like elements employed in respective embodiments is omitted for sake of brevity.

In the embodiment shown, the patient placement prioritization model development module 104 can include clustering component 202, attribute identification component 204, model generation component 206, and attribute weighting component 208. In one or more embodiments, the patient placement prioritization model development module 104 can evaluate how to prioritize patient placement requests for beds at a medical facility to optimize patient flow and patient care by initially distinguishing between different types of patient placement requests represented in the historical data 116. In this regard, the clustering component 202 can be configured to evaluate the historical data 116 to identify different clusters of patient placement requests, wherein the requests included each cluster are associated with one or more common elements.

In some embodiments, the clustering component 202 can be configured to cluster the historical patient placement requests as a function of a combination of a type of bed and a type of medical service associated with the request. For example, inpatient medical facilities can apply different rules and policies regarding fulfilling patient placement requests based on the type of medical service needed by the patient and type of bed needed by the patient in association with provision of the medical service to the patient. These rules and policies can control for example, where a patient can be treated, when the patient should be treated, who can treat the patient, medical supplies needed for treating the patient, protocols for treating the patient, and the like. For example, most hospitalizations involve one or more procedures, which can range from simple vaccinations to complex surgical procedures. The principal procedure is the procedure that is performed for definitive treatment (e.g., an appendectomy), but procedures can also be performed to make a diagnosis (e.g., tissue samples or exploratory surgery). Hospitalizations usually involve more than one procedure, which can often involve transferring of a patient different medical units and beds at which the different procedures are performed. In this regard, different types of beds are used for different types of situations and patients conditions and they serve different purposes of treatments. In some implementations, the different types of beds and the different types of medical services provided by the healthcare facility that can be used to classify patient placement requests can be predefined. For example, different medical services include defined medical procedures (e.g., in accordance with current procedural code terminology (CPT)), such as a type of surgery, an imaging study procedure, a type of physical examination, and the like. In some implementations the bed type associated with a patient placement request can be categorized or determined based on a required level of care requested for the patient in association with the placement request.

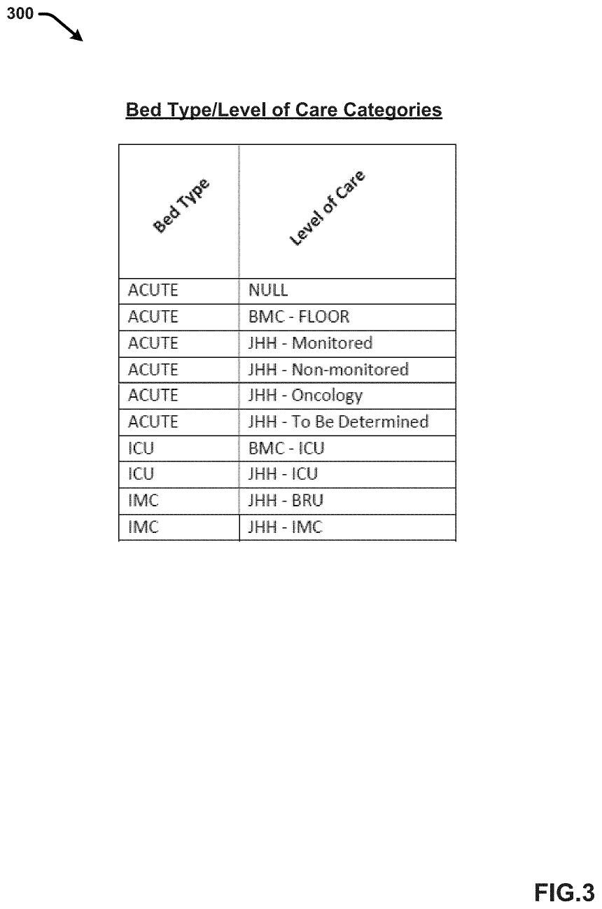

For example, FIG. 3 presents a table 300 that defines some example bed types categorized by level of care that can be used to classify different types or clusters of bed requests in accordance with one or more embodiments of the disclosed subject matter. In the embodiment shown, three different bed types are listed, including an acute care unit (ACUTE) bed type, an intensive care or intensive care unit (ICU) bed type, and an intermediate care unit (IMC) bed type. Different levels of care are respectively associated with one of the defined bed types. For example, levels of care characterized as “(NULL)” (Level of care not specified), “Monitored” (telemetry based monitoring), “Non-monitored,” and “To-be-determined,” are all respectively considered as requiring an acute bed type. Also in the embodiment shown, the levels of care include ICU which is associated with an ICU bed type, and the levels of care including “Brain Rescue Unit” and “IMC (Intermediate Care)” are associated with an IMC bed type.

Accordingly, with reference again to FIG. 2, in one or more embodiments the clustering component 202 can cluster the patient placement requests represented in the historical data 116 into different clusters or types of patient placement requests, wherein each cluster or type of patient placement request comprises requests with a distinct combination of bed type and medical service. The number of different patient placement request types or clusters can vary based on the medical facility or facilities represented in the historical data. For instance, in one example implementation in which the historical data 116 comprises historical performance and operational data for a hospital or hospital system with various different medical specialty units, the number of different patient placement requests identified in the historical data 116 may range from 100 to 200 distinct combinations. In some embodiments, the clustering component 202 can also cluster based patient placement requests based on other criteria, such as but not limited to, medical service line associated with the request, different medical care units to which patient placement request are assigned, classification information that classifies requests with a predefined priority status, demographic information regarding the patient for which the requests are placed, patient insurance categories, predicted length of stay, and the like.

In various embodiments, the attribute identification component 204 can identify attributes associated with the different types/clusters of patient placement requests that influence how (e.g., when and where) the respective patient placement requests are fulfilled. The terms attributes, parameters, factors, features, characteristics, criteria and the like are used throughout the subject disclosure interchangeably. In this regard, the attribute identification component 204 can identify attributes, parameters, factors etc. that may impact how patient placement requests are prioritized. For example, the attributes can include but are not limited to: request characteristics (e.g., urgency, level of care, hospital service etc.), policy details (e.g., unit preference, patient hierarchy schemes, etc.), location details (e.g., current location, transfer location, etc.), occupancy levels, bed availability, and the like.

In some embodiments, the attribute identification component 204 can employ one or more machine learning techniques (e.g., supervised, unsupervised, neural networks, etc.) to identify direct and indirect (or hidden) correlations between various parameters that influence when placement requests are fulfilled relative to timing of initial placement of the requests and other pending patient placement requests. For example, some attributes that may impact when a patient placement request is filled can relate to the requirements of the request, state of the patient, level of urgency of the request, preferences of the patient, insurance held by the patient, duration of time in which patient will occupy the bed (e.g., based on duration of time to perform a procedure and allow the patient to recover before the patient can be released), and the like. The attribute identification component 204 can also employ one or more machine learning techniques to identify one or more contextual parameters associated with patient placement requests that influence when and how the requests are fulfilled. In this regard, contextual attributes related to the current context surrounding a request, such as the context of the healthcare facility and/or the specific unit or units where the patient can be placed (e.g., to receive the requisite care), and in some implementations the forecasted context of the healthcare facility and/or suitable unit or units, can impact when the request is fulfilled. For example, some example contextual factors may can include current bed availability in preferred units, current bed availability in alternate units, bed EVS status, number of patient placement requests for the same bed ahead of the patient, availability of appropriate clinicians to treat the patient, location of the patient and the location of the requested bed, availability of appropriate medical supplies to treat the patient, predicted changes to bed availability, predicted amounts and types of future placement requests to be received within an upcoming window of time, and the like.

The attribute identification component 204 can also employ one or more machine learning techniques to identify direct and indirect correlations between parameters that influence how different placement requests are fulfilled. In this regard, attributes that may affect how a patient placement request is fulfilled can include those which influence what specific medical unit the patient is assigned in association with fulfillment of the request, as well as parameters that influence specific clinical and/or financial outcomes associated with treating the patient based in association with fulfilment of the patient placement request and the like.

In other embodiments, attributes or parameters that may (or have been previously determined to) influence when and how different patient placement requests are fulfilled can be predefined (e.g., and stored in memory 108 or another suitable and accessible memory device). For example, one or more attributes or parameters that are relevant to patient placement decisions may be determined based on defined policies of the healthcare facility, personal experience with the bed management process and interviews with the bed managers, patients, clinicians, medical facility personnel, and the like. With these embodiments, the attribute identification component 204 can be configured to identify extract and extract the predefined attributes for each patient placement request included in the historical data. In other embodiments, the attribute identification component 204 can be provided with some predefined attributes known to influence when and how patient placement requests are fulfilled and also employ machine learning to identify additional attributes in the historical data that have some correlation to when/how patient placement requests are fulfilled.

In some implementations, the attribute identification component 204 can be configured to generate an indexed and cleansed data structure based on the historical data 116 that includes information identifying the historical patient placement requests represented in the historical data 116 as organized by request type/cluster. The indexed data structure can also include attribute information, for each (or in some implementations one or more) placement request that defines values for the different attributes that have been determined (e.g., via machine learning and/or as predefined) to influence when and how the patient placement request was fulfilled.

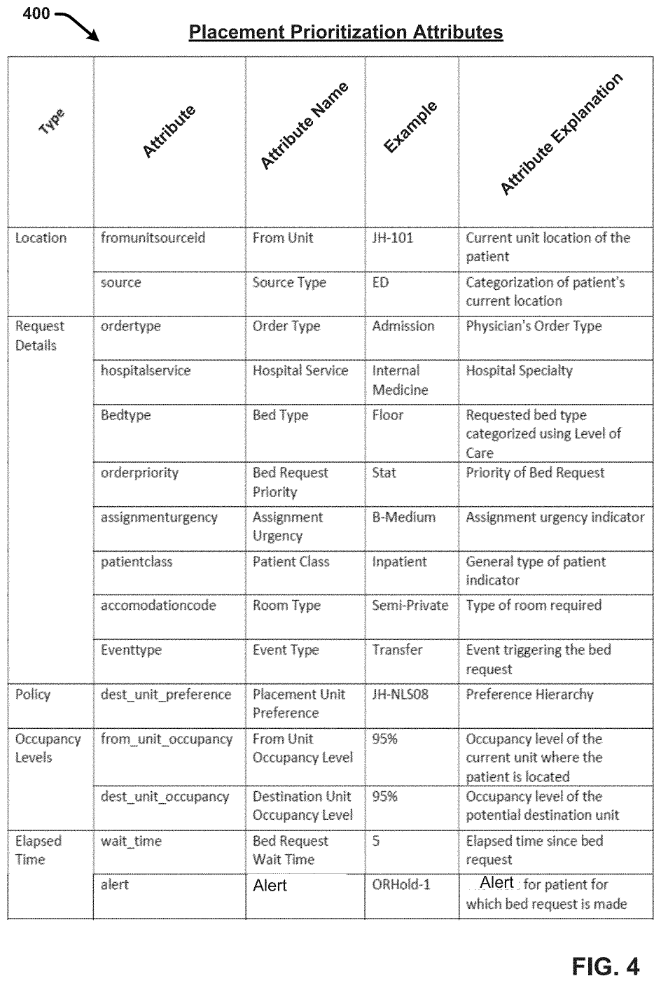

FIG. 4 presents a table 400 identifying example attributes that may influence, or have been previously determined to influence, when and how patient placement requests are fulfilled in accordance with various embodiments described herein. In some embodiments, one or more of the attributes included in table 400 can be identified in the historical data 116 by the attribute identification component 204 using one or more machine learning techniques. In other embodiments, one or more of the attributes included in table 400 can be predefined as known attributes that influence fulfillment of patient placement requests (e.g., at a particular healthcare facility, at various healthcare facilities in general, etc.).

In this regard, table 400 identifies 15 different attributes that can influence fulfilment of patient placement requests. The attributes are respectively divided into five different type categories, including location, request details, policy, occupancy levels and elapsed time. Table 400 further includes information identifying the computer readable attribute term used for the respective attributes (provided in the second column), the natural language attribute name (provided in the third column), an example of the attribute (provided in the fourth column), and an explanation of the attribute (provided in the fifth column) For example, the term “fromunitsourceid” corresponds to a location attribute that represents the unit from which a patient associated with a patient placement request is currently located. The location attributes also include a source type attribute that corresponds to a categorization of the patient's current location. With reference to the natural language attribute names (the third column), the request details attributes include order type, hospital service, bed type, bed request priority, assignment urgency, patient class, room type, and event type. A single policy attribute is provided that includes the placement unit preference. The occupancy level attributes include the occupancy level of the unit from which the patient will be transferred and the occupancy level of the destination unit. The elapsed time attributes include the bed request wait time, and alert (which corresponds to any type of notification or alert regarding a status of a request). For example, in some implementations, alerts can be generated by an analytics system when there are delays beyond a configured threshold. These delays can be in the care process, transition process or any other administrative process. For instance, if there is an alert for a ED patient boarding in a ED bay and to be moved to an inpatient unit, the disclosed placement prioritization techniques can account for these alerts and prioritize appropriately. Various levels of alerts can be prioritized differently. For an alert for a patient waiting for 30 minutes can be prioritized differently than patient waiting for 30 hours. It should be appreciated that the attributes provided in table 400 are merely exemplary and that various implementations of the disclosed subject matter can include additional and/or alternative attributes.

With reference again to FIG. 2, based on the various attributes, parameters, factors, etc., associated with the different patient placement requests that have been determined to influence or potentially influence when and how one or more of the different patient placement requests are fulfilled, the model generation component 206 can be configured to develop one or more placement prioritization models 120 that correlate the manner in which one or more of the above described attributes influence a patient placement prioritization scheme for fulfilling the patient placement requests that achieves optimal patient flow (e.g., by minimizing request fulfilment delay times, reducing congestion and bottlenecks, and the like) and optimal patient care (e.g., in terms of quality of care provided, achieving of optimal clinical outcomes, etc.). In particular, the model generation component 206 can be configured to evaluate the historical data 116 using one or more machine learning techniques, to identify optimal patient placement patterns that result in optimal patient flow and patient quality of care. The model generation component can further evaluate the optimal patient placement patterns to learn how patient placement requests are prioritized in accordance with the optimal patient placement patterns, and more particularly, the manner in which certain attributes/contextual conditions associated with the respective requests impact how the respective requests are prioritized to achieve the optimal patient placement pattern. The model generation component 206 can further develop one or more placement prioritization models 120 that correlate the various attributes and the contextual factors associated with a particular patient placement request with a prioritization scheme for fulfilling the request (that achieves optimal patient flow and care). In this regard, a patient placement prioritization scheme can define a relative priority order for fulfilling two or more pending patient placement requests based on attributes/characteristics associated with the requests and a contextual state of the healthcare facility (e.g., wherein the contextual state of the healthcare facility can account for one or more contextual factors regarding occupancy levels, predicted occupancy levels, number of pending requests, availability of clinicians, availability of requisite medical supplies, etc.).

In various embodiments, this machine learning analysis involve learning how one or more attributes associated with a particular patient placement request influence how to prioritize the request relative to other pending requests and given a current state of the healthcare facility, to achieve optimal patient flow and care for the entire healthcare facility. According to these embodiments the patient placement prioritization model development module 104 can include attribute weighting component 208 to determine how one or more of the different attributes, parameters, etc. (e.g., attributes listed in table 400 for example, attributes determined by the attribute identification component 204, and the like) play a role in the priority level/order to be given to a particular patient placement request, wherein the priority level controls patient placement in a manner that achieves optimal patient flow and patient care. For example, the attribute weighting component 208 can be configured to determine, using machine learning analysis of the historical data 116, the manner in which different attributes associated with a particular placement request impact a priority level to be given to the request that results in fulfilling the request in a manner that achieve optimal patient flow and patient care. In this regard, the attribute weighting component 208 can be configured to employ one or more machine learning techniques in association with analysis of the historical data 116 to determine the relative importance of different attributes and/or relationships between different attributes associated with a particular patient placement request, to a priority level of the request. The model generation component 206 can further employ the learned relative importance weightings of and/or relationship between the different attributes associated with a particular patient placement request in association with developing one or more placement prioritization models 120 that models a relative priority level for fulfilling the request based on the attributes that will likely achieve optimal patient flow and patient care.

In one or more embodiments, the model generation component 206 can generate one or more placement prioritization models 120 that correlate the various attributes associated with different patient placement requests to an optimal priority scheme for fulfilling the requests (e.g., that achieves optimal patient flow and patient care) using a weighted sum method with a linear additive utility function. This method can combine both quantitative and qualitative information associated with the respective requests. In accordance with these embodiments, the linear additive utility function can correlate different attributes associated with pending patient placement requests (and the context associated with the requests) to prioritization scores for the respective requests. The prioritization scores can thus correlate priority levels of the respective patient placement requests relative to one another in view of achieving optimal patient flow and care. In this regard, the prioritization scores can reflect the manner in which various clinical and operational factors, unique to each request and the context of each request, impact patient flow and care. Higher prioritization scores for example can indicate a higher priority of a patient placement request relative to lower prioritization scores, wherein fulfilment of patient placement requests with higher prioritization scores before patient placement requests with lower prioritization scores is likely to achieve better patient flow and care.

In accordance with these embodiments, the model generation component 206 can develop a different placement prioritization model 120 for each (or in some implementations, one or more) different type/cluster of patient placement request identified by the clustering component 202. In this regard, the placement prioritization models 120 can respectively be configured to determine a priority order for patient placement requests of a same type/cluster that are competing for the same bed. For example, bed requests with same specialty and bed type along with other attributes can be defined as the collection of alternatives that are available to the bed manager as choice set. Thus, in some embodiments, the model generation component 206 can generate different patient placement prioritization models, (or different patient placement model parameters/parameter weight values), for every (or in some implementations one or more) valid and distinct combination of hospital service and bed type. In this regard, the specific attributes and/or the manner in which the attributes impact a prioritization score for a patient placement request can vary for each type/cluster of patient request.

In some embodiments, the model generation component 206 can also be configured to generate different prioritization models for each (or in some implementations one or more) medical unit at the healthcare facility. With these embodiments, leaning and model development can be carried out for each distinct combination of hospital service and bed type within each specific medical unit. In this regard, each medical unit can be associated with a plurality of different prioritization models that are tailored to that medical unit, wherein each periodization model within the medical unit is configured to a distinct combination of medical service and bed type. For example, a patient is often a candidate for medically appropriate placement in several different units. When determining how to prioritize placing the patient, the disclosed system can be configured to run the distinct (unit specific) machine learning based scoring prioritization model/algorithm for each medical unit where the patient can be placed. As a result, a different prioritization score can be generated for the request for each of the different medical units.

The weights for the respective attributes/contextual factors accounted for in the linear additive utility function for each type of patient placement request can be estimated based on analysis of the historical data 116 using one or more machine learning techniques. For example, in one embodiment, the model generation component 206 can employ a Guided Unbiased Interaction Detection and Estimation (GUIDE) regression tree to learn placement patterns from historical data 116. Once the placement patterns are learned, the attribute weighting component 208 can estimate the importance scores for each attribute and determine the relative weights from these importance scores. Learning and model development can be carried out for every (or in some implementations one or more) type of patient placement request characterized by a distinct combination of hospital service and bed type to generate a different placement prioritization model 120 for each distinct combination. These resultant models and/or the parameters and parameter weights of these models can further be stored in the model database 118 with information identifying the type of patient placement request that they are respectively developed for (e.g., the different bed type and medical service combination associated with each model).

In this regard, in one or more embodiments, using the weighted sum method with the linear additive utility function, the model generation component 206 can frame the patient placement prioritization problem with the following weighted sum function:

Prioritization Score P(rᵢ)=Σⱼ₌₁^{m}wⱼsᵢⱼ i=1, . . . ,N∀i∈Z Equation 1,

wherein the subscript i is an index for a finite number of patient placement requests N,
wherein i belongs to Z which is defined as a set of data comprising patient placement requests of a same type (e.g., a request type defined by a specified hospital service and bed type),
wherein wⱼ is percentage weight of j^{th} factor among m factors (e.g., attributes such as those represented in table 400 and the like), and wherein sᵢⱼ is the normalized factor value of the j^{th} factor for request i. In accordance with Equation 1, an additive utility function is applied by assuming wⱼ≥0 and Σⱼ₌₁^{m}w=1.

In accordance with Equation 1, the model generation component 206 can normalize factor values (and weights) using the following linear scale transformations:

$$\begin{matrix}{s}_{ij} = {max}_{new} - ( {x}_{ij} - {max}_{old} ) \cdot \frac{{max}_{new} - {min}_{new}}{{max}_{old} - {min}_{old}} , & Equation 2 \\ {s}_{ij} = {min}_{new} + ( {x}_{ij} - {min}_{old} ) \cdot \frac{{max}_{new} - {min}_{new}}{{max}_{old} - {min}_{old}} , & Equation 3\end{matrix}$$

wherein max_{new} and min_{new} are the defined thresholds for normalized factor values.

Equation 2 is applied for criteria where more is worse, (e.g. the higher tier levels in the preference hierarchy. In other words, the less preferred the unit is for a given hospital service and bed type, the lower the normalized factor values). For example, Table 1 below demonstrates example normalized factor values for medical units associated with different rank or tier levels.

TABLE 1

| | | Factor Value |
|---|---|---|
| Unit | Rank or Tier Level | (Normalized) |
| JH-NLS08 | 1 | 100 |
| JH-NLS03 | 2 | 67 |
| JH-NLS06 | 3 | 34 |
| JH-NLS07 | 4 | 1 |

Likewise, Equation 3 is applied for criteria where more is better, (e.g. wait time). For example, patients waiting longer can be prioritized over patients waiting relatively shorter periods of time. When normalized factor values are developed, higher factor values are assigned for higher wait times. For example, Table 2 below demonstrates example normalized factor values for different wait times for requests with a same service (general medicine) and bed type (Acute).

TABLE 2

| | | Wait Time | Factor Value |
|---|---|---|---|
| Service | Bed Type Attribute | (In minutes) | (Normalized) |
| General Medicine | Acute | 1 | 1.00 |
| General Medicine | Acute | 1440 | 50.48 |
| General Medicine | Acute | 2880 | 100 |

The attribute weighting component 208 can estimate or determine the weights (wᵢ) for the respective attributes/parameters of Equation 1 above from the historical data 116 using one or more machine learning techniques (e.g., supervised machine learning, unsupervised machine learning, neural networks, etc.). For example, in one exemplary embodiment, the model generation component 206 and/or the attribute weighting component 208 can employ a regression tree based method to facilitate determining the relevant attributes and attributes weights for the prioritization model (e.g., Equation 1) as applied to each type of patient placement request. A regression tree method can be used to obtain model parameter values by recursively partitioning the X vector space and fitting a prediction model within each partitioned vector space. In accordance with this embodiment, the model generation component 206 can further model the patient prioritization problem as a regression tree problem in accordance with Equation 4:

$$f ( X ) = {\sum }_{m} {y}_{m} I ( X ∈ {D}_{m} ) , \qquad Equation 4$$

wherein X is the matrix of regressor variables, γₘ are the estimated values of the time to pre-assign in region Dₘ. Equation 4 assumes bed managers will maximize their utility by prioritizing and pre-assigning patients. Hence, minimizing time to pre-assign is defined as utility maximizing behavior by the bed managers. Time to pre-assign is defined as time from bed request to unit assignment. In this regard, with respect to Equation 4, γₘ can be a prediction model such as piecewise constant regression model, I(⋅) is an indicator function returning 1 if its argument is true and 0 otherwise. The disjoint data partitions of the training data D (e.g., the historical data 116) are represented by Dₘ such that ∪ₘDₘ=D and ∩ₘDₘ=Ø. In this regard, the model generation component 206 can determine the relevant model parameters by minimizing the mean squares error criteria as follows:

$$\frac{1}{n} {\sum }_{i}^{n} {( {t}_{i} - r ( β , {x}_{i}^{′} ) )}^{2} , \qquad Equation 5$$

wherein tᵢ is the time to preassign a request, wherein xᵢ^{t} is a p-dimensional vector of observed attributes that relate to request i, and wherein β is the corresponding vector of coefficients.

The attribute weighting component 208 can further employ the GUIDE supervised learning method to estimate the importance scores of the attributes. For example, GUIDE has some useful properties such as negligible selection bias through its use of residual analysis (chi-square tests), automatic handling of missing data, sensitivity to local and pairwise interaction between regressor variables, ability to handle categorical variables including ordinal categorical variables with relative ease, and importance ranking along with identification of unimportant variables. These properties facilitate generation of placement prioritization models that provides a truthful description of the historical data and validate some of the rules and reasoning applied by the bed managers for prioritizing bed placements. In this regard, in implementations in which the attribute weighting component 208 employs GUIDE, the attribute weighting component 208 can estimate the importance score of a variable as follows:

IMP(x)=Σₜ\sqrt{n(t)}χ₁²(x,t) Equation 6,

wherein x is the regressor variable or selected attribute, t is the intermediate node in the regression tree, n(t) is the sample size in node t and χ₁²(x,t) is the upper quantile q(x,t) of the chi-squared distribution with one degree of freedom.

In one or more embodiments, the attribute weighting component 208 can employ Equation 6, (also referred to herein as the GUIDE algorithm), to obtain the importance scores for the identified relevant attributes for each (or in some implementations one or more) of the different types/clusters of patient placement requests. The attribute weighting component 208 can then determine the relative weights for each attribute from the unscaled importance scores using the following equation:

$${w}_{j} = \frac{IMP ( {x}_{j} )}{{\sum }_{i}^{m} IMP ( {x}_{j} )} . \qquad Equation 7$$

In this regard, in accordance with the weighted sum prioritization model (e.g., Equation 1), attributes that contribute to a higher prioritization level/score for a particular type of request can be associated with a greater relative/unscaled weight than attributes that contribute to a lower prioritization level/score.

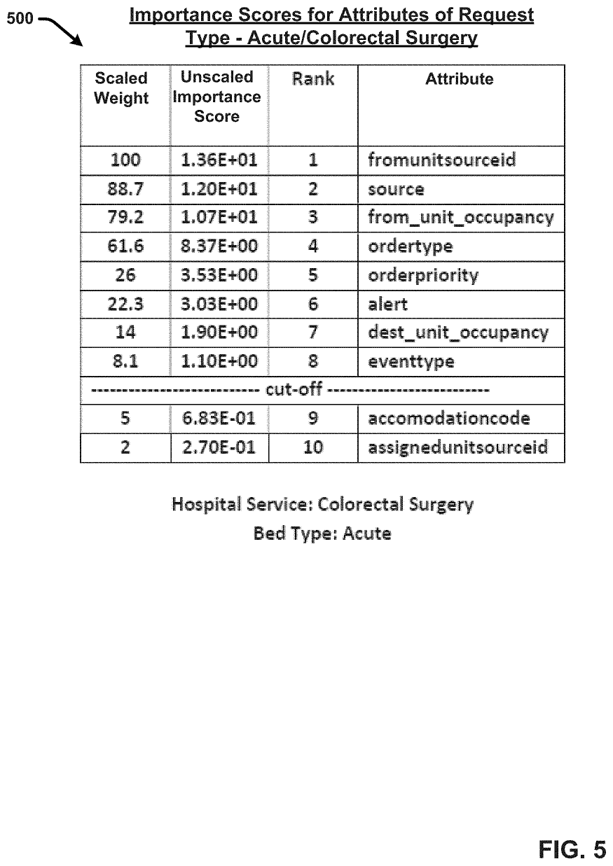

FIG. 5 presents a table 500 with some example scaled and unscaled scores for attributes associated with one type of patient placement request. For example, in the embodiment shown, the type of patient placement request is characterized as “acute/colorectal surgery,” (e.g., bed type=acute, medical service=colorectal surgery). The scaled weight and unscaled importance scores shown in table 500 respectively represent scaled and unscaled attribute scores/weights that can be determined by the attribute weighting component 208 using Equations 7 and 6, respectively. The respective attributes including “fromunitsourceide,” “source,” “from_unit_occupancy,” “ordertype,” “orderpriority,” “alert,” “dest_unit_occupance,” “eventype,” “accomodationcode,” and “assignedunitsoruceid,” are ranked from highest to lowest importance based on their relative scaled weights/unscaled importance scores. These example attributes are described with reference to table 400. In the embodiment shown, the attributes having weights/scores below a defined threshold (e.g., “accomodationcode,” and “assignedunitsoruceid,”) can be identified and excluded from the prioritization model (e.g., Equation 1), for the specific type of patient placement request.

With reference again to FIG. 2, in one or more embodiments, the model generation component 206 and/or the attribute weighting component 208 can perform supervised machine learning in accordance with Equations 4-7 for each (or in some implementations one or more) of the different types/clusters of patient requests to determine the respective weights and normalized factor values for each of the factors that may influence or have been determined to influence how the respective requests are prioritized. The relevant attributes and associated weights for each type of patient placement request can further be stored in the model database 118 in association with placement prioritization models 120 developed for each type of patient placement request (e.g., modeled using Equation 1 or the like). These parameters are critical elements of the scoring model (e.g., Equation 1) that can be used to generate a prioritization score for each unfulfilled bed request in real-time.

Because the manner in which same attributes influence how different types of patient placement requests are prioritized can vary, the weights associated with respective attributes as included in the different placement prioritization models can also vary. In some implementations, different prioritization models for different types of requests can be associated with different subsets of attributes (e.g., different subsets of attributes represented in table 400). In other implementations, attributes that are irrelevant or substantially irrelevant to the prioritization level of a particular type of patient placement request can be given zero weight.

In some embodiments, for a given type of patient placement request characterized by a specific medical service and a specific bed type/required level of care, the patient may be placed in two or more predefined medical units to receive the appropriate care. With these embodiments, the request can be associated with information defining a designated medial unit as a preferred unit and/or rank the alternate units in order of preference. For example, information defining the specific units where each defined type of patient placement request can be fulfilled, including information defining unit preference hierarchy, can be predefined and stored in memory 108 (or another suitable memory accessible to the computing device 102). In accordance with these embodiments, a prioritization score can be determined for the request for every preferred or alternate unit. The requests can further be sorted from high to low score within each unit. Placement requests can further be ranked on their sorted scores. Rankings along with the availability of beds in their preferred or alternative units are then used to prioritize the bed requests. For example, based on the patient's prioritization scores for the respective medical units where the patient can be placed (e.g., that meet the patient's care requirements), the patient may achieve recommended potential placement in more than one unit at a time. In this case, if one of the optional medical units is known to be a preferred unit, the preferred unit may be selected if it is one in which the patient was ranked for potential placement. However, in other implementations, the system may recommend, (and/or an operator may also choose), to place the patient in a less preferred unit in situations where the less preferred unit is less highly in demand. For example, this allows for the unit with a long list of patients needing placement to be used for patients for whom this more available unit is not an option.

In one implementation of these embodiments, the specific patient placement prioritization model (included in the placement prioritization models 120) developed for the specific type of patient placement request can be used to determine a prioritization score for the request relative to each of the possible units (e.g., the preferred unit and the one or more alternate units). In this regard, the prioritization scores determined for the request can vary for the different units based on variances between model parameter values that can vary from unit to unit (e.g., the destination unit occupancy level). In another implementation, the model generation component 206 can generate different placement prioritization models for each of the different units. For example, the model generation component 206 can generate different placement prioritization models based on a combination of bed type, medical service, and medical unit. In this regard, in implementations in which the placement prioritization models 120 are respectively characterized by Equation 1, wherein the parameter weights are tailored for each type of request, the placement prioritization models 120 can further include a defined set of weights for the parameters for each combination of bed type, medical service, and medical unit. The attribute weighting component 208 can determine the parameter weights using machine learning analysis of the historical data 116 in accordance with Equations 4-7, as described above.

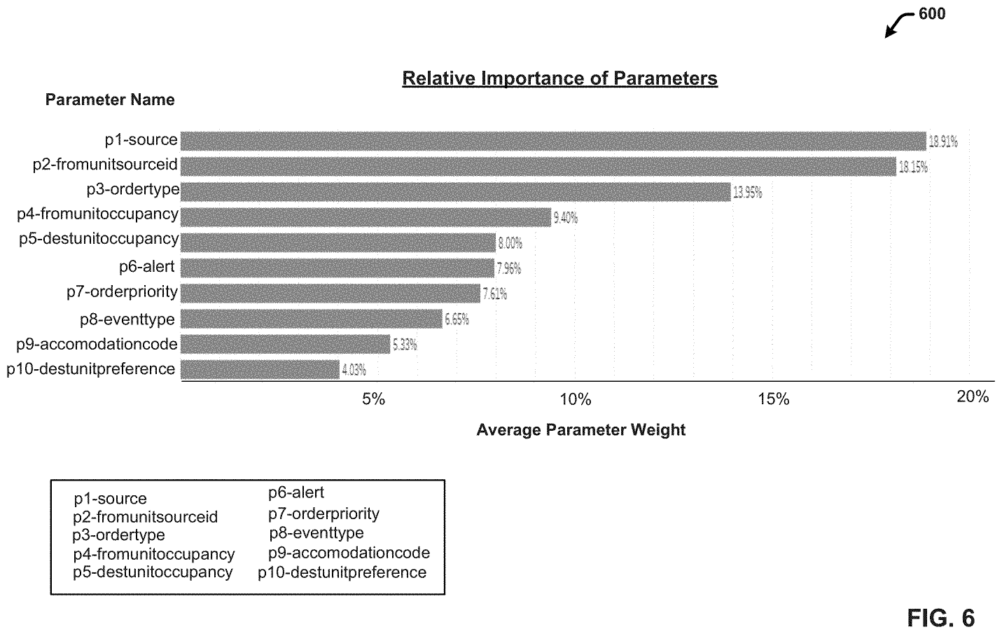

FIGS. 6-9 present charts 600, 700, 800 and 900, respectively, demonstrating example weights of different parameters reflective of their relative importance in association with determining priority levels of various types of patient placement requests in accordance with one or more embodiments of the disclosed subject matter. The example parameters evaluated in FIGS. 6-9 are respectively identified as follows: p1-source, p2-fromunitsourceid, p3-ordertype, p4-fromunitoccupancy, p5-destunitoccupancy, p6-alert, p7-orderpriority, p8-eventtype, p9-accomodationcode, and p10-destunitpreference. The parameters p1-p10 are respectively described infra with reference to FIG. 4 and table 400 (e.g., the parameters p1-p10 correspond to attributes presented in table 400). The example weights of parameters shown in chart 600, 700, 800 and 900, respectively reflect analysis of a historical training data set comprising historical bed management data, performance data, operational data and the like, representative of 177 distinct types of patient placement requests at hospital. The example weights of the parameters shown in FIGS. 6-9 were respectively determined via the attribute weighting component 208 in accordance with embodiments described herein. Repetitive description of like elements employed in respective embodiments is omitted for sake of brevity.

With reference to initially to FIG. 6, chart 600 demonstrates example weights of the ten different parameters p1-p10 reflective of their relative importance in association with determining priority levels of various types of patient placement requests in accordance with one or more embodiments of the disclosed subject matter. In accordance with this example implementation of the patient placement prioritization model development module 104, chart 600 reveals that p1, the originating source of the request (e.g. ICU), p2, the current unit of the patient (e.g. Medical ICU Unit), and p2, the order type (e.g. Admission) are factors that are weighted higher than the rest. Ports of entry are synonymous with sources and held accountable for moving patients in a timely manner Certain ports of entry are primary sources of patients and employees at these sources collaborate with bed managers to ensure patients are transitioned smoothly for their care and this is reflected through the weightings determined via the attribute weighting component 208 using the machine learning techniques described herein.

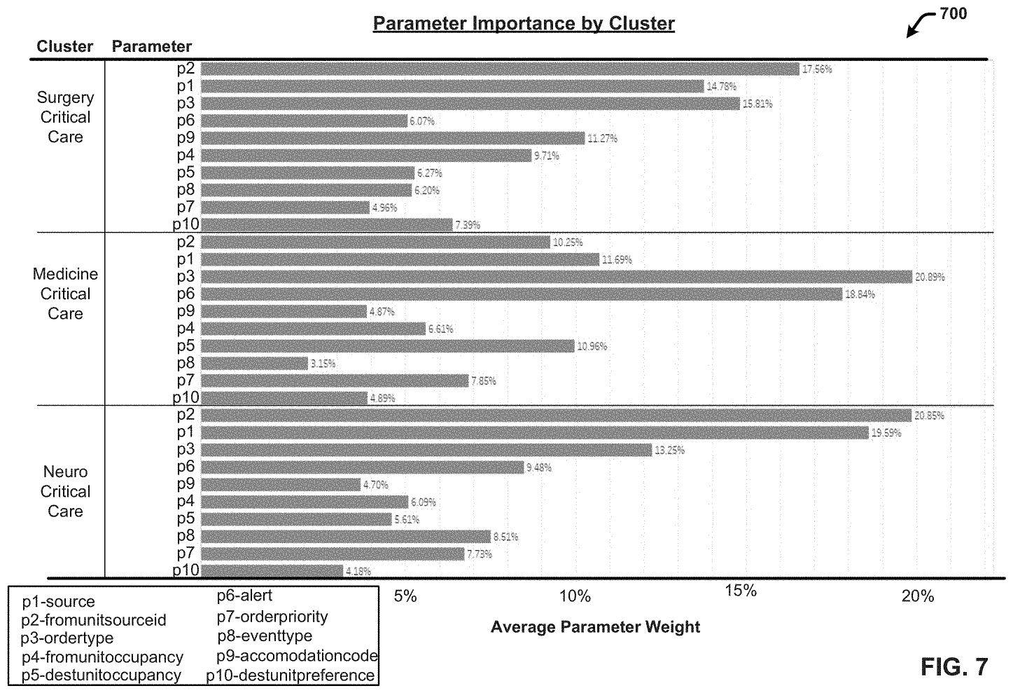

FIG. 7 presents a chart 700 demonstrating example average weights of different parameters reflective of their relative importance in association with determining priority levels of patient placement requests in the critical care unit, in accordance with one or more embodiments of the disclosed subject matter. In accordance with chart 700, the critical care unit is divided into three different subunits, including surgical critical care, medicine critical care, and neuro critical care. As shown in chart 700, the average parameter weights for p1-p10 vary relative to these three different types of critical care units. In this regard, in one implementation, the attribute weighting component 208 can associate each subunit with a different type of patient placement request and thus a different placement prioritization model 120 and determine specific weights for the parameters relative to each subunit. For example, each the three different critical care subunits are considered associated with a distinct cluster of patient placement requests. Chart 700 demonstrates that in accordance with this example implementation of the patient placement prioritization model development module 104, the that current location of the patient (p2) and order type (p3) are weighted more for placing patients in the surgery and neurology critical units relative to the medicine units. At the same time, order type (p3) and patient flow alerts (p6) are weighted more for placing patients in the medicine critical units.

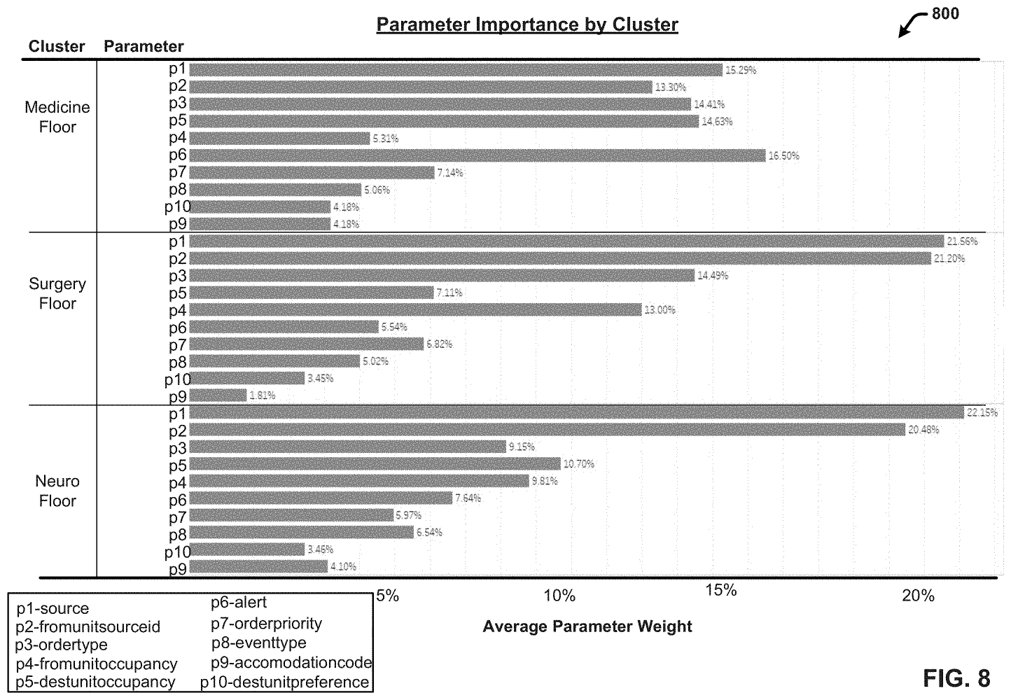

FIG. 8 presents a chart 800 demonstrating example average weights of different parameters reflective of their relative importance in association with determining priority levels of patient placement requests in the floor unit, in accordance with one or more embodiments of the disclosed subject matter. In accordance with chart 800, the floor unit is divided into three different subunits, including medicine floor unit, surgery floor unit, and neuro floor unit. As shown in chart 800, the average parameter weights for p1-p10 vary relative to these three different types of floor units. In this regard, in one implementation, the attribute weighting component 208 can associate each floor subunit with a different type of patient placement request and thus a different placement prioritization model 120 and determine specific weights for the parameters relative to each floor subunit. For example, each the three different critical care subunits are considered associated with a distinct cluster of patient placement requests. Chart 800 demonstrates that in accordance with this example implementation of the patient placement prioritization model development module 104, the requesting source (p1), current patient location (p2), and destination unit occupancy levels (p5) are factors weighted higher than the rest for placing patients to the neuro floor unit. On the other hand, the requesting source (p1), current patient location (p2), and order type (p3), are the most critical factors for placing patients to surgical floor unit. In addition, several factors such as requesting source (p1), current patient location (p2), destination unit occupancy levels (p5), and patient flow alerts (p6) are the most critical factors for placing patients to medicine floor units.

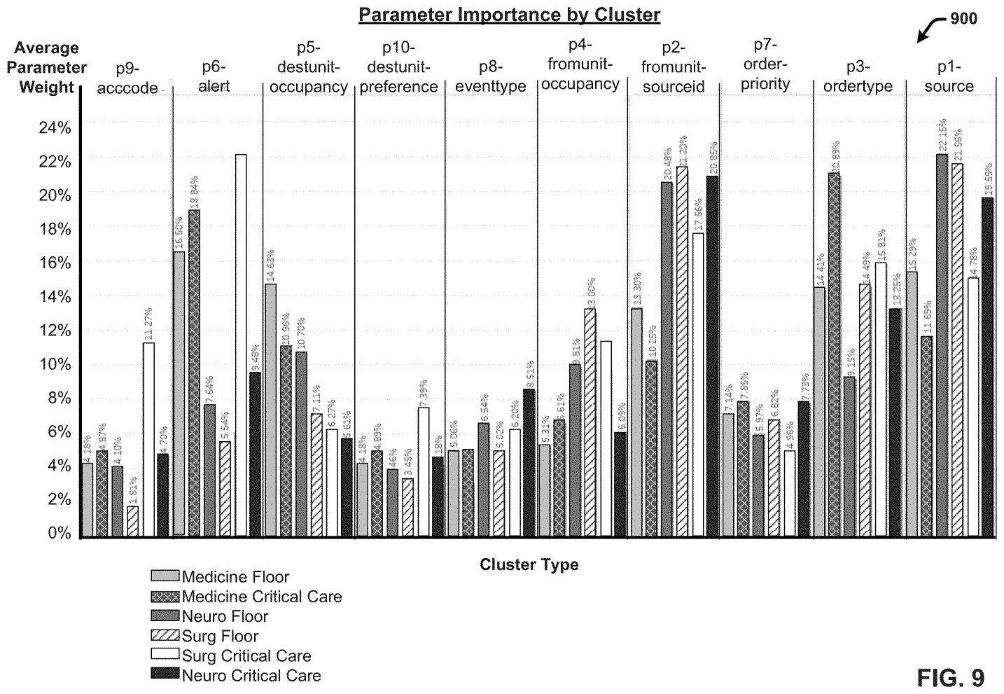

FIG. 9 presents a chart 900 comparing weighting of factors amongst the critical care and floor unit clusters for medicine, neurology and surgery service line, in accordance with one or more embodiments of the disclosed subject matter. In accordance with chart 900, different parameter weights for parameters p1-p10 are compared as arranged per cluster type characterized by medical care unit type. In this regard, in one implementation, the attribute weighting component 208 can associate the different unit types, including medicine floor unit, medicine critical care unit, neuro floor unit, surgery floor unit, surgery critical care unit, and neuro critical care unit, with a different type of patient placement request and thus a different placement prioritization model 120 and determine specific weights for the parameters relative type of medical unit. In accordance with this example, implementation, chart 900 demonstrates that the parameter weights can vary greatly by medical unit. For example, it is interesting to note that patient flow alerts have highest average weight for medicine critical care and floor clusters.

Referring now to FIG. 10, presented is a block diagram of an example, non-limiting system 1000 that provides for employing a multifactorial, machine-learning based prioritization framework for optimizing patient placement in real-time, in accordance with one or more embodiments of the disclosed subject matter. System 1000 presents some additional example components of the patient placement prioritization module 112 that can perform different operations associated with applying the one or more placement prioritization models 120 to facilitate determining how to prioritize patient placement requests in a live healthcare setting. In one or more embodiments, system 1000 is subsystem of system 100 (e.g., system 100 can include system 1000, and vice versa). Repetitive description of like elements employed in respective embodiments is omitted for sake of brevity.

In the embodiment shown, the patient placement prioritization module 112 can include request component 1002 selection component 1004, importer component 1006, and scoring component 1008. As described supra, in one or more embodiments, the patient placement prioritization module 112 can monitor one or more medical facility systems 122 (e.g., bed management systems, scheduling systems, patient monitoring systems, etc.) in real-time over the course of operation of the medical facility to identify and extract information relevant to facilitating determining how to prioritize inpatient patient placement requests at the healthcare facility. In this regard, the request component 1002 can be configured to monitor one or more medical facility systems 122, such as bed management systems and the like, to identify placement requests 124 made at the healthcare and real-time state data 126 that is relevant to determining how to manage fulfilling the patient placement requests. For example, the request component 1002 can be configured to identify placement requests 124 that include requests for bed assignments for newly admitted patients, requests for bed reassignments for currently admitted patients, requests for bed transfers for currently admitted patients, and the like.

In various embodiments, the placement requests 124 received at the healthcare facility and identified by request component 1002 can include or be associated with information identifying or indicating a type of the request. For example, in implementations in which the patient placement requests are clustered (e.g., via the clustering component 202) based on bed type and medical service, the placement requests 124 can be associated with information identifying the bed type and medical service associated with the request. In some implementations, the patient placement requests can also be associated within information identifying one or more medical units where the request can be fulfilled (e.g., a preferred unit, one or more alternate units, etc.). In other implementations, the patient placement prioritization module 112 can determine what unit or units where a request can be fulfilled using predefined information (e.g., stored in memory or otherwise accessible to the patient placement prioritization module 112) associating request types with one or more defined medical units at the healthcare where they can be fulfilled.

Based on identification and/or reception of a new placement request 124, the selection component 1004 can be configured to identify and select an appropriate patient placement prioritization model from the one or more placement prioritization models 120 included in the model database 118 for applying to the request. For example, in embodiments in which different placement prioritization models 120 are respectively tailored to specific types of requests characterized by a distinct combination of bed type and medical service, the selection component 1004 can select a specific placement prioritization model from the placement prioritization models that is tailored to the bed type/medical service combination associated with the new patient placement request. In this regard, the different placement prioritization models can include different parameters, parameter weights, and/or different algorithms (different than Equation 1) for relating the relevant parameters to a prioritization score. In another example, in embodiments in which different placement prioritization models 120 are respectively tailored to specific types of requests characterized by a combination of bed type, medical service and medical unit, the selection component 1004 can determine one or more medical units where the patient can be placed. In this regard, if the request can be fulfilled in two (or more) medical units, the selection component 1004 can select two (or more) placement prioritization models from the models included in the model database 118, one for each type of medical unit and request type.

In some implementations, based on identification of a new patient placement request placed at the healthcare facility (e.g., as new requests are received in real-time over the course of operation of the healthcare facility), the importer component 1006 can import, extract, or otherwise receive additional relevant information associated with the request, (e.g., in addition to information regarding requested bed type and medical service), provided by the one or medical facility systems 122 regarding details of the request and real-time state data 126 relevant to the request. For example, the importer component 1006 can be configured to import/extract information from the one or more medical facility systems 122 regarding defined input parameters of the specific patient placement prioritization model or models selected by the selection component 1004 for application to the request. For instance, the importer component 1006 can import, retrieve or otherwise receive information regarding the patient for the which the request is made, location information identifying the current location of the patient and the categorization of the patient's current location, policy information regarding preference hierarchy polices for the unit or units where the patient can be place, request details (e.g., an order type, associated with the request, a hospital specialty associated with the request, a pre-classified bed request priority, an assignment urgency indicator regarding an assignment urgency of the request, a preferred medical unit, a patient class, a room type, an event type, etc.), and the like. With respect to relevant real-time state data 126, the importer component 1006 can extract information regarding other pending patient placement requests of the same type and/or for the same medical unit or units where the patient can be place, bed availability, current occupancy levels in medical units where the patient is currently located, occupancy levels in the one or more units where the patient can be placed, duration of elapsed time since the request was made, alerts for the patient associated with the request, information regarding locations and availability of clinicians, information regarding locations and availability of medical supplies, and other types of conditional factors regarding a current state of the healthcare facility that can influence how the patient placement request is prioritized for fulfilment in accordance with the one or more placement prioritization models selected for application to the request.

For example, with reference again to FIG. 5, table 500 identifies 8 attributes (above the cut-off scaled weight) that were identified as relevant to determining the priority score of a patient placement request type acute/colorectal surgery. In this regard, the attributes represented in the patient placement prioritization model developed for the request type acute/colorectal surgery can include those 8 attributes identified in table 500. According to this example, when a patient placement request is received of request type acute/colorectal surgery, the importer component 1006 can identify and extract information from the one or more medical facility systems 122 providing the attribute values for those top 8 attributes identified in table 500.

Accordingly, with reference again to FIG. 10, for every (or in some implementations, one or more) patient placement request that is received at the healthcare facility that system 1000 is used to monitor and evaluate, the patient placement prioritization module 112 can immediately (e.g., within a few seconds), identify the request, select the appropriate placement prioritization model from the model database 118 for applying to the request, and identify and extract relevant input information from the one or more medical facility systems 122 for input to the selected placement prioritization model based on the attributes/parameters represented in the placement prioritization model. The scoring component 1008 can further employ the selected prioritization model to determine a prioritization score for the received patient placement request that reflects a priority level of the request. In this regard, the scoring component 1008 can provide the identified and extracted values for the parameters represented in the patient placement model (e.g., which can include attributes related to the patient, the request, the context of the healthcare facility, etc.) as input to the patient placement prioritization model to determine the patient placement prioritization score. For example, in embodiments in which the patient placement prioritization model is characterized by Equation 1, the prioritization score can represent a weighted sum of a defined set of attributes, wherein the attributes and/or the weights for the attributes were determined based on machine learning analysis of historical bed management data for the healthcare facility (e.g., by the attribute identification component 204, the model generation component 206 and/or the attribute weighting component 208).

Thus, in one or more embodiments, the patient placement prioritization information 128 can include a prioritization score determined for a patient placement request received at the medical facility. The patient placement prioritization module 112 can further generate and report the prioritization score in the patient placement prioritization information 128 in real-time or substantially real-time as the request is placed at the healthcare facility. In this regard, the processing time associated with identifying and/or receiving the patient placement request by the request component 1002, selecting the appropriate prioritization model for the request by the selection component 1004, extracting the input parameter values for the prioritization model by the importer component 1006, and applying the prioritization model using the input parameter values to generate the prioritization score by the scoring component 1008, can be a few seconds or less. Accordingly, in various embodiments, the patient placement prioritization information 128 can include one or more prioritization scores determined for one or more patient placement request received at the healthcare facility. For example, the patient placement prioritization information 128 can identify one or more newly received patient placement requests and/or one or more pending patient placement requests at the healthcare facility, information identifying the patient placement requests (e.g., a request identifier, a patient name, etc.), and the prioritization score determined for the request.

In some implementations, the patient placement prioritization information 128 can also identify a specific medical unit where a placement request can be fulfilled. With these implantations, the prioritization score can vary depending on the medical unit. For example, the prioritization model can include one or more attributes associated with the destination medical unit, wherein the values and/or weights of the one or more attributes can vary depending on the destination unit (e.g., the occupancy level of the destination unit can vary, the weight given to a certain order type associated with the destination unit can vary, etc.). In another example, different prioritization models can be developed for each medical unit and request type. According to this example, the attributes included in the model and/or the weights of the attributes can vary for the different prioritization models associated with the different medical units. In this regard, in implementations in which a placement request can be fulfilled in two or more medical units, the patient placement prioritization information 128 can include prioritization scores determined for the request as applied to each of the two or more medical units. The prioritization scores for the same request as applied to the two or more medical units can vary. For example, the prioritization score associated with fulfilment of the request in medical unit A can be higher than the prioritization score associated with fulfilment of the request in medical unit B, and vice versa.

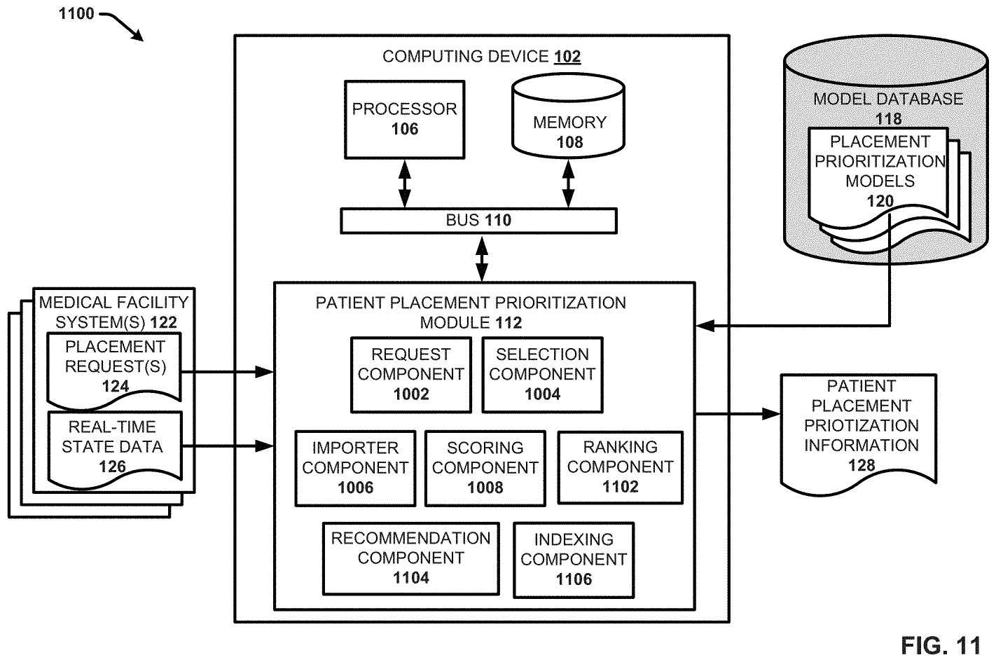

FIG. 11 illustrates a block diagram of another example, non-limiting system 1100 that provides for employing a multifactorial, machine-learning based prioritization framework for optimizing patient placement in real-time, in accordance with one or more embodiments of the disclosed subject matter. System 1100 include same or similar features and functionalities as system 1000 with the addition of ranking component 1102, recommendation component 1104, and indexing component 1106 to the patient placement prioritization framework. Repetitive description of like elements employed in respective embodiments is omitted for sake of brevity.

The prioritization score determined for a received patient placement request by the scoring component 1008 represents a priority level of the request that can be used to determine how to prioritize fulfilling the request relative to other pending patient placement requests for the same bed. In this regard, each type of placement request 124 received at the healthcare facility can be associated with a specific bed type required for fulfilling the request and information describing details of the request identifying or indicating one or more medical units where the request can be fulfilled. In one or more embodiments, the ranking component 1102 can be configured to identify all pending (unfulfilled) patient requests for same bed type per medical unit where the requests can be fulfilled, and rank the respective requests based on their respective priority scores. For example, all pending requests for an acute bed type that can be assigned to medicine floor medial unit can be identified and ranked according to their priority scores. The recommendation component 1104 can further recommend a prioritization scheme for fulfilling the requests based on their ranking and the number of available beds in the medical unit. With these embodiments, the ranking component 1102 can determine information regarding bed availability and pending patient placement requests from the real-time state data 126 monitored at the one or more medical facility systems 122. The patient placement prioritization module 112 can further determine the patient placement prioritization scores for the respective pending patient requests using the techniques described with reference to FIG. 10 and system 1000.

For example, assuming three acute care bed types are available in the medicine floor unit and there a five pending patient placement requests that can be assigned to the beds, the recommendation component 1104 can recommend assigning the top three placement requests associated with the top three highest prioritization scores to the beds. Accordingly. in some embodiments, the patient placement prioritization information 128 can include information that identifies pending placement requests for a particular bed type in a particular medical unit and their respective prioritization scores. The placement requests can further be ordered or ranked based on their prioritization scores (e.g., highest to lowest). In addition, in some implementations, the patient placement prioritization information 128 can include information identifying the number of available beds in the particular medical unit of the particular bed type and/or recommendation information indicating what requests to assign to the available beds (as determined by the recommendation component 1104). For example, in one implementation, the patient placement prioritization information 128 can identify one or more pending patient placement requests recommended for assigning to one or more available beds in one or more medical units.

In some embodiments in which a patient placement request can be fulfilled in two or more medical units, the recommendation component 1104 can also determine where to assign the patient based on the number of available beds in the respective units, and the ranking of the request relative to other pending placement requests for the available beds. For example, in assuming the following example scenario: A patient placement request for patient John Doe can be fulfilled in either medical unit A or medical unit B. Medical unit A has one available bed and the ranking of the patient placement request in medical unit A is third on the list (e.g., meaning two patient placement requests have higher prioritization scores than John Doe's request). However, medical unit B has two available beds and the ranking of the patient placement request relative to unit B is second highest on the list. In accordance with this example scenario, the recommendation component 1104 can recommend assigning patient John Doe to the available bed in unit B, because this would result in minimizing the delay time between reception of the request and fulfillment of the request. According to this example, the recommendation component 1104 can generate patient placement prioritization information 128 that includes an assignment recommendation recommending patient John Doe to the available bed in medical unite B.

However, in some embodiments in which a patient placement request can be assigned to two or more medical units, the request can be associated with information identifying one or the medical units as a preferred unit. In these scenarios, determining whether to place a patient in the first available bed in an alternate medical unit or wait for an available bed in the preferred unit can require some additional consideration. With these scenarios, in some implementations, the recommendation component 1104 can allow the bed manager to choose what to do and forgo providing a recommendation merely based on minimizing delay time. In other implementations, the recommendation component can employ one additional criteria to determine what choice to make. For example, the additional criterial can include a deeper analysis of the details of the request (e.g., urgency of the request, policies associated with the request), the prioritization score of the request, one or more contextual factors associated with the current state of the medical facility, information regarding predicted timing of bed availability in the preferred unit, and the like, to further determine whether to proceed to recommend placing the patient in the alternate bed unit or waiting until a bed opens up in the preferred unit. In some implementations, the recommendation component 1104 can employ artificial intelligence and one or more machine learning techniques based on analysis of patterns in the historical data 116 to facilitate determining which decision to make.

In association with determining prioritization scores for a group of pending placement requests for a same bed type and/or medical unit, in one or more embodiments, the scoring component 1008 can regularly update prioritization scores for the respective requests as parameters values used to determine the respective prioritization scores can change over time (e.g., contextual parameters associated with the current state of the healthcare facility and/or the requests, alerts associated with one or more requests). In this regard, the scoring component 1008 can be configured to regularly update prioritization scores determined for one or more pending placement requests included in a group of pending placement requests competing for a same bed/medical unit based one or more defined events, such as but not limited to: reception of a new request for the same bed/medical unit, change in status of a request in the group, change in one or more parameters values associated with one or more of the requests, passage of a defined amount of time (e.g., every 20 minutes update scores), and the like prioritization scores.

In some embodiments, to facilitate efficiently evaluating, scoring, and updating prioritization scores for all (or in some implementations one or more) pending placement requests at the healthcare facility, the indexing component 1106 can be configured to generate an indexed data structure for all (or in some implementations, one or more) requests received and evaluated by the patient placement prioritization module 112. The indexed data structure can be stored in memory 108 and organize information relevant to managing fulfilment of the respective requests based on their prioritization scores. For example, in one or more implementations, the indexed data structure can identify, for each received request, the timing of reception of the request, details associated with the request, the prioritization model or models selected for the request and applied to the request, values for the parameters of the prioritization models, the current prioritization score or scores associated with the request, and the like. The indexing component 1106 can further monitor changes to relevant information for the respective requests included in the indexed data structure based on the real-time state data 126 or other information provided by the medical facility systems 122. The indexing component 1106 can further regularly update the information associated with respective request in the indexed data structure as appropriate. For example, the indexing component 1106 can monitor and update information regarding the status of a request (e.g., fulfilled, on hold, etc.), alerts associated with the request, elapsed time since the request was received, changes to the prioritization score or scores determined for the request, and the like.

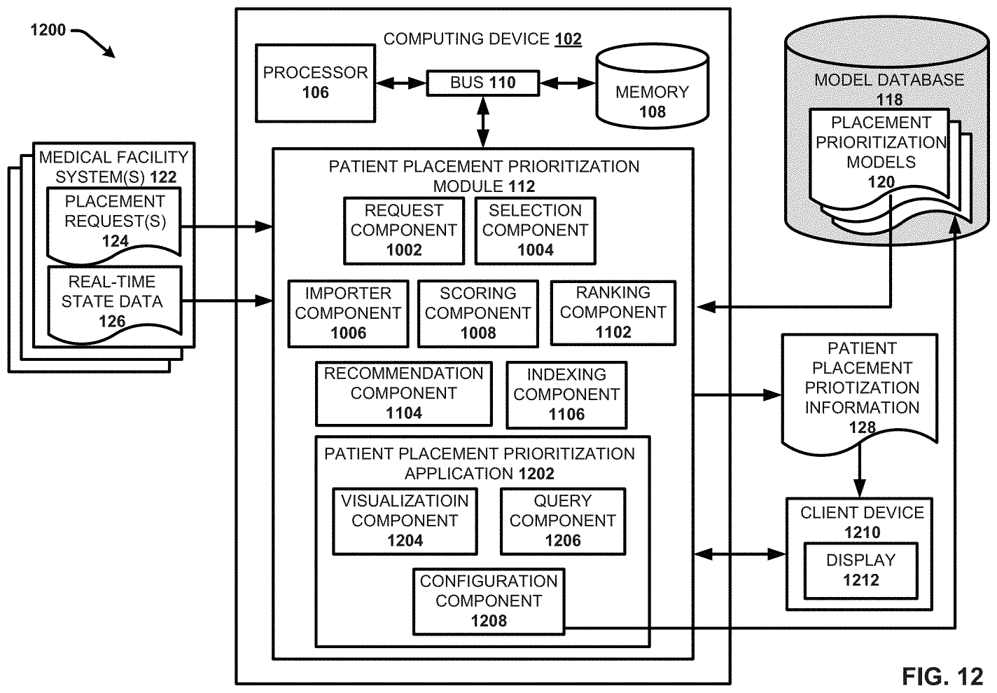

FIG. 12 illustrates a block diagram of another example, non-limiting system 1200 that provides for employing a multifactorial, machine-learning based prioritization framework for optimizing patient placement in real-time, in accordance with one or more embodiments of the disclosed subject matter. System 1200 include same or similar features and functionalities as system 1100 with the addition of a client device 1210 and the addition of a patient placement prioritization application 1202 to the patient placement prioritization module 112. Repetitive description of like elements employed in respective embodiments is omitted for sake of brevity.

In various embodiments, the patient placement prioritization module 112 can generate and provide patient placement prioritization information 128 to actual bed managers, clinicians, patients, or other suitable entities in real-time to facilitate managing bed assignments at the healthcare facility in real-time. For example, the patient placement prioritization module 112 can be configured to provide the patient placement prioritization information 128 to one or more client/user devices (e.g., client device 1210) associated with bed managers and/or other appropriate entities for rendering via a display 1212 and/or another suitable output device (e.g., in an audible format via a speaker, as sensory or haptic feedback, and the like) to facilitate bed management decisions in a live hospital environment. For instance, in some implementations, the client device 1210 can include a display monitor, a television, an Internet enabled television or the like that can provided at one or more locations throughout the healthcare facility accessible to the bed mangers, clinicians or the like. In other implementations, the client device 1210 can represent one or more internal computers used by the bed managers/clinicians at the healthcare facility in association with managing bed assignments. In another implementation, the client device 1210 can represent one or more personal devices used by the bed managers/clinicians or other appropriate entities, such as personal tablet computers, smartphones, and the like. Other suitable client devices can include but are not limited to: a desktop computer, a laptop computer, a television, a personal digital assistant (PDA), a heads-up display (HUD), an augmented reality (AR) device, a virtual reality (VR) device, a wearable device, and the like.

In some embodiments, the patient placement prioritization module 112 can be configured to simply provide the patient placement prioritization information 128 for rendering at the one or more client devices in real-time in a well-organized and structured format. The patient placement prioritization module can further regularly provide updated patient placement prioritization information as it changes over the course of operation of the healthcare facility. In this regard, the patient placement prioritization information 128 can help bed managers understand how to best prioritize patient placements to increase placement efficiency, decrease placement delay times, and provide quality care for the patients.

In various additional embodiments, the patient placement prioritization module 112 can provide an interactive, patient placement prioritization application 1202 that can provide various interactive features and functionalities associated managing bed placement at an inpatient healthcare facility. For example, in one or more embodiments, system 1200 (and other systems described herein) can embody, be incorporated into and/or be employed by, a centralized hospital command center. In this regard, the hospital command center can be or include a network accessible system the facilitates hospital operations management by combining engineering with advanced analytics to better manage patient experience, patient flow and capacity. With these embodiments, the features and functionalities of the patient placement prioritization module 112 can be accessed and interfaced with by one or more entities associated with the hospital (e.g., via a client device 1210) using the patient placement prioritization application 1202. In this regard, the patient placement prioritization application 1202 can provide a centralized bed management function that facilitates real-time understanding of bed supply and demand across the entire hospital enterprise. The information provided by the patient placement prioritization application 1202 can be current, interactive and transparent across the entire hospital to help clinicians make most informed decisions for the patients and their care while having a holistic view of the state of the healthcare facility to make more informed decisions.

The patient placement prioritization application 1202 can provide various interactive features and functionalities associated with accessing, viewing and interacting with the patient placement prioritization information 128 and information imported by the importer component for respective requests and indexed and regularly updated by the indexing component 1106 regarding details of all pending placement requests. The patient placement prioritization application 1202 can also provide users various interactive features and functionalities associated with accessing, viewing and interacting with other relevant information (e.g., relevant real-time state data 126) provided by one or more of the medical facility systems 122 that can facilitate bed managers in association with fulfilling patient placement requests. For example, such additional relevant information can include bed availability and EVS status information, occupancy information regarding current occupancy levels throughout the healthcare facility, information regarding current status and locations of medical supplies needed in association with fulfilment or requests, information regarding locations and activities of clinicians, policy information regarding polices of the healthcare facility to be followed in association with fulfilment of the requests, scheduling information, forecasted information regarding predicted bed availability and occupancy levels and the like.

Accordingly, the patient placement prioritization application 1202 can incorporate a large number of elements critical for prioritizing patient placements and provide information and services that can significantly improve the decision-making capabilities of the bed managers. For example, the patient placement prioritization application 1202 can not only provide patient placement prioritization information 128, but also incorporate information regarding bed availability, EVS status, bed requests, occupancy levels, competing demand, forecasts and much more in a single application. The patient placement prioritization application 1202 can thus integrate and transform real-time disparate raw data provided by the one or more medical facility systems 122 into useful actionable information for decision making.

In the embodiment shown, the patient placement prioritization application 1202 can include visualization component 1204, query component 1206 and configuration component 1208. The visualization component 1204 can be configured to generate and/or provide an interactive graphical user interface (GUI) including visualizations that facilitate evaluating and managing patient placements in real-time. The visualizations can integrate real-time information regarding pending patient placement requests, prioritization scores determined for the pending patient placement requests, recommendation information regarding how to prioritize bed assignments (e.g., determined via recommendation component 1104), bed availability, occupancy levels, EVS status, and potentially other contextual information regarding a current state of the medical facility that can be relevant to determining when and where a patient is placed. In this regard, the visualization component 1204 can present prioritization scores for pending patient placement requests to the bed managers in a structured and meaningful way.

For example, in some embodiments, the patient placement prioritization application 1202 can facilitate generating a GUI that can be displayed at one or more client devices (e.g., a client device 1210 via a display 1212) associated with bed managers (or other appropriate entities). Via the interactive GUI, the patient placement prioritization application 1202 can allow users (e.g., bed managers) to view patient placement requests grouped by medical service line, grouped by medical service, grouped by specialty, grouped by request type (e.g., a distinct bed type/medical service combination), grouped by medical unit, or another criterion. In some implementations, the patient placement prioritization application 1202 can allow users to provide input identifying two or more criteria associated with patient placement request for filtering and organizing into subsets of the available information for viewing into manageable parts. In association with providing information identifying patient placement requests grouped by one or more defined criteria, the patient placement prioritization application 1202 can also provide detailed information associated with the requests regarding prioritization scores determined for the requests, prioritization schemes recommended for fulfilling the requests based on the prioritization scores (e.g., by recommendation component 1104). The patient placement prioritization application 1202 can also incorporate other details associated with the requests such attributes used to determine the prioritization scores for the respective requests, information organized and indexed by the indexing component 1106, and the like. For example, in association with providing information identifying a request and a prioritization score for the requests, the visualization component 1204 can provide information identifying the patient, the request time (e.g., duration of time passed since reception of the request), the originating source of the requests, a specialty associated with the request, a current location of the patient, an order priority of the request, a level of care associated with the request, an event type associated with the request, and the like. The visualization component 1204 can further update any dynamic information included in the GUI and/or associated visualizations in real-time as it changes over the course of operation of the healthcare facility.

In some implementations, the GUI and/or associated visualizations (e.g., charts, tables, images, etc.) can be rendered on one or more video walls associated with the healthcare facility, networked visualization solutions, mobile device displays and the like. For example, in one implementation the patient placement prioritization application 1202 can be provided as a tile service of a hospital command center system that employs and/or comprises one or more components of system 1200. The user facing interfaces generated and/or provided by the visualization component 1204 can facilitate efficient navigation of comprehensive information about pending patient placement requests at a medical facilitate and the options available for fulfilling them. In this regard, the patient placement prioritization application can provide interactive functions that allow users to view real-time information regarding the pending patient requests from a holistic view of the entire hospital and further drill down to more granular view of requests associated with different medical units and individual request.

The query component 1206 can facilitate a query function of the patient placement prioritization application 1202. In this regard, the query component 1206 can facilitate querying the information available to the patient placement prioritization application 1202, including information monitored and indexed by the indexing component 1106, patient placement prioritization information 128, and information provided by the one or more medical facility systems 122 (e.g., placement requests 124 and real-time state data 126). For example, the query component 1206 can be configured to receive query terms via a GUI generated and/or provided to a client device 1210 via the visualization component and return filtered and organized information for rendering via the GUI based on the query terms. For instance, the query component 1206 can generate subsets of information related to patient placement requests, prioritization scores, medical units, bed availability, occupancy levels, patient flow, contextual information regarding locations and activities of clinicians, medical supplies, and the like. The visualization component 1204 can further organize the subsets of information into a visualization for display via the GUI that facilitate efficiently evaluating the information.

The configuration component 1208 can provide a configuration function of the patient placement prioritization application 1202 that allows users to adjust one or more parameters and/or parameter weights defined for one or more placement prioritization models 120. In this regard, the configuration component 1208 can access the placement prioritization models 120 and present information regarding the parameters and parameter weights defined for one or more selected models via a configuration interface. The configuration component 1208 can allow bed managers or other appropriate entities to provide input that changes weights and/or normalized factor values of the one or more (selected) placement prioritization models 120 via the configuration interface. In some implementations, the updated model parameters and/or weights can be stored with the original patient placement prioritization model as a variation of the original model. In other implementations, the updated patient placement model with the modified parameters and/or parameter weights can replace the original patient placement model stored in the model database 118.

In one or more embodiments, the patient placement prioritization application 1202 can provide the information and associated interactive services disclosed herein to entities (via their respective client devices 1210) via a suitable network accessible platform. For example, in some implementations, the client device 1210 can include an application (e.g., a web browser) for retrieving, presenting and traversing information resources on the Internet, an intranet, or the like. According to these implementations, the patient placement prioritization application 1202 can provide processed information and interactive tools to end users via a website platform that can be accessed using a browser provided on their respective client devices (e.g., client device 1210). In another implementation, the patient placement prioritization application 1202 can provide processed information and interactive tools to users as a mobile application, a thin client application, a thick client application, a hybrid application, a web-application and the like. With these implementations, one or more components of the patient placement prioritization application 1202 can be located at an application server (e.g., the computing device 102) and/or the client device 1210. In this regard, the client device 1210 can be communicatively coupled to the application server (e.g., the computing device 102 or another suitable real or virtual computing device at which one or more components of the patient placement prioritization application 1202 can be accessed), either directly or via one or more networks, (not shown). Such network(s) can include wired and wireless networks, including but not limited to, a cellular network, a wide area network (WAN, e.g., the Internet, an Intranet), a local area network (LAN), a personal area network (PAN) and the like. Still in other implementations, one or more components of the patient placement prioritization application 1202 can be provided at the client device 1210 and accessed directly in an offline mode.

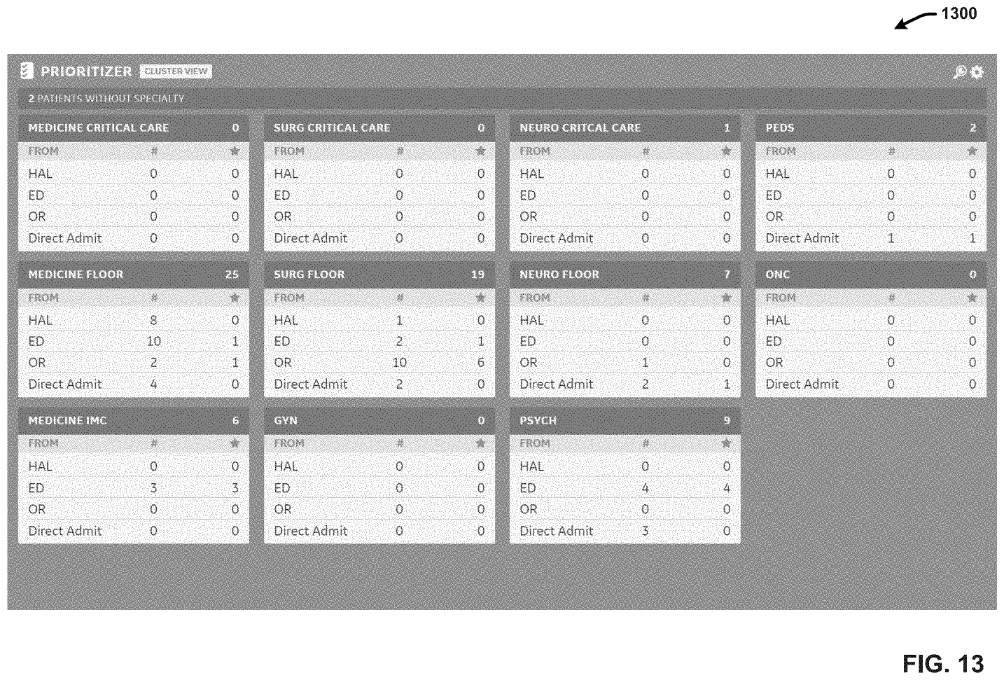

FIGS. 13-23 present various graphical user interfaces (GUIs) and/or visualizations that demonstrate various features and functionalities of the patient placement prioritization application 1202 in accordance with embodiments described herein. In one or more embodiments, the GUIs and/or associated visualization presented in FIGS. 13-23 can be generated and/or provided by the visualization component 1204 and rendered at one or more client devices (e.g., client device 1210) associated with one or more entities involved with bed management at a healthcare facility. In addition, the various prioritization scores and schemes represented in FIGS. 13-23 can be determined via the by the patient placement prioritization module 112 (e.g., via the selection component 1004, the scoring component 1008, the ranking component 1102, and/or the recommendation component 1104) in accordance with the techniques described herein. Various features and functionalities of the patient placement prioritization application 1202 are now described with reference to FIGS. 13-23. Repetitive description of like elements employed in respective embodiments is omitted for sake of brevity.

FIG. 13 presents an example GUI including an aggregate cluster view visualization 1300 of patient placement requests in accordance with one or more embodiments described herein. The aggregate cluster view visualization 1300 presents an aggregate view of the placement information visually grouped as clusters. Each cluster is associated with a set of hospital service and bed type combination, which can be pre-defined by the bed managers as part of the solution configuration. For the best user experience and visualization, cluster names can include names of medical service line and bed type or it could just display the medical service line name. This can be configured by users to reduce the number of clusters and highlight the clusters with certain attributes like high volume. For example, bed managers are often interested in evaluating information regarding bed requests of given service line. This information can be presented in both an aggregate and detailed format. The aggregate cluster view visualization 1300 provides an aggregate view of patient placement requests in eleven different pre-defined service lines including: medicine critical care, surgery critical care, neurology critical care, pediatrics (PEDS), medicine floor, surgery floor, neurology floor, oncology (ONC), medicine intermediate care ward (IMC), gynecology (GNC), and physiology (PSYCH). In the embodiment shown, each service line cluster is provided within a distinct table/box. The total number of pending patient placement requests associated with each service line is provided in the upper righthand corner of the respective tables. For example, the medicine critical care cluster has zero pending placement requests, the medicine floor service line has 25 pending placement requests, the intermediate medicine care (IMC) cluster has 6 pending placement requests, and so on.

The tables for each cluster respectively include three columns. The column with the “FROM” heading identifies the current source type of the patient (e.g., wherein source type refers to a categorization of the patient's current location). For example, in the embodiment shown, four source types are identified including Inter Hospital Transfers (HAL), Emergency Department (ED), Operating Rooms (OR), or (Direct Admits). The middle column with the number or hashtag symbol (#) heading identifies the respective number of pending patient placement requests originating from the different source types HAL, ED, OR and Direct Admin. The star column can identify the number of patients prioritized for placement within the cluster, organized by source type. For example, with respect to the medicine floor, one patient coming from the ED unit is prioritized for placement in the medicine floor and one patient coming from the OR unit is prioritized for placement in the medicine floor. In accordance with one or more embodiments of the subject disclosure, the patient prioritization schemes identified in the aggregate cluster view visualization 1300 were determined by the recommendation component 1104 based on prioritization scores respectively associated with all pending requests associated with a same medical service line cluster.

In some embodiments, the respective cluster tables presented in the aggregate cluster view visualization 1300 can be selected (e.g., clicked on or otherwise selected) to view a detailed view of the specific requests that fall within a cluster of interest.

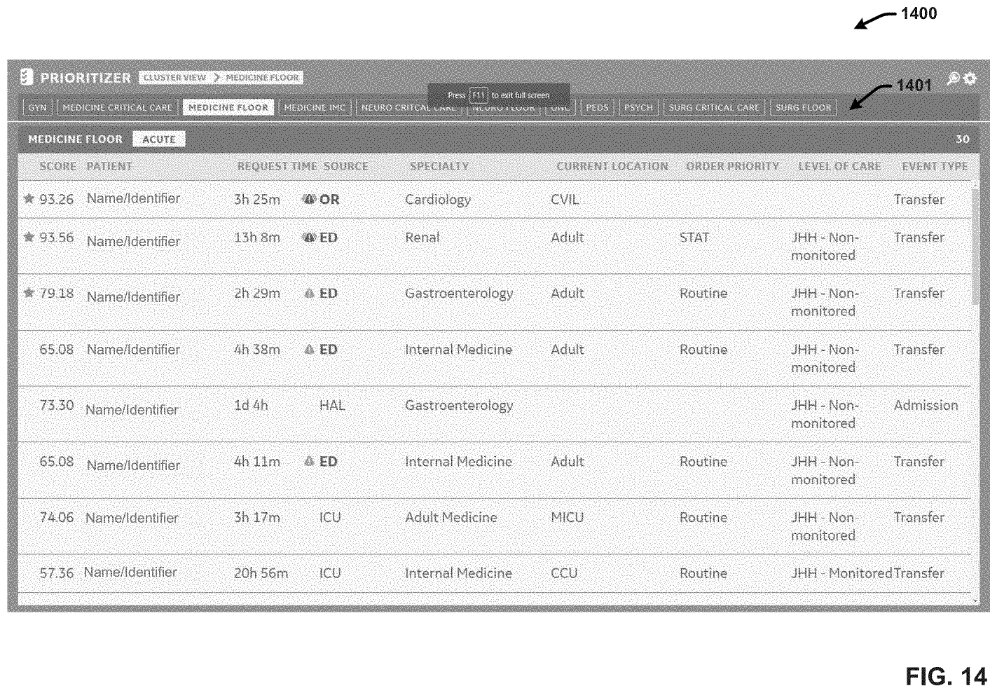

For example, FIG. 14 presents an example GUI including a detailed cluster view visualization 1400 that provides a detailed view of the bed requests for acute beds types in the medicine floor service line, in accordance with one or more embodiments described herein. In one implementation, the detailed cluster view visualization 1400 can be provided (e.g., by the visualization component 1204) based on selection of the medical floor service line table as presented in the aggregate cluster view visualization 1300. The detailed cluster view visualization 1400 can include a service line toolbar 1401 that allows for selecting and switching between viewing placement requests within different service lines. For example, in the embodiment shown, the medicine floor service line is shown in white to indicate that it is currently selected. The detailed cluster view visualization 1400 can also include identify the certain bed type within the selected service line represented by the requests in the table. For example, in the embodiment shown, only one bed type, the ACCUTE bed type represented.

The detailed cluster view visualization 1400 provides various details of each request to the medicine floor unit for the acute bed type arranged in a scrollable table format. For example, the table provides several columns with detailed information for each request, including the prioritization score, the patient, the request time (representing the amount of time passed since the request was placed), the source of the request, the specialty type of the medical service associated with the request, the current location of the patient, the order priority, the level of care, and the event type. In the embodiment shown, the respective requests are arranged or sorted in descending order based on their respective prioritization scores.

A star symbol is used throughout the various aggregate and detailed placement request visualizations presented herein to indicate the requests recommended for prioritizing to available beds. For example, in the embodiment shown, the requests with the top thee highest prioritization sores are associated with star symbols to indicate these requests are recommended (e.g., by the recommendation component 1104) for assigning to the available beds first. In this regard, a star symbol can indicate that for a patient placement request having a given hospital service and bed type combination, a bed is available in either a preferred unit or an alternate unit and the rank of the request is less or equal to the number of available beds in that unit.

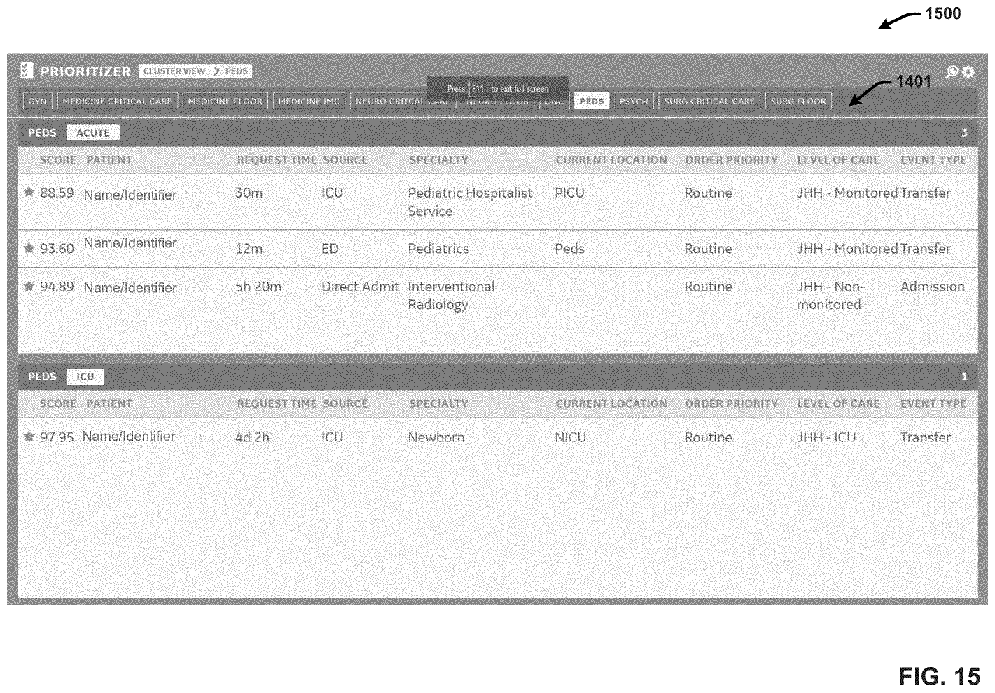

FIG. 15 presents an example GUI including another detailed cluster view visualization 1500 that provides a detailed view of the bed requests within a specific medical service line in accordance with one or more embodiments described herein. For example, the detailed cluster view visualization 1500 presents requests in the PEDS service line. Similar to the detailed cluster view visualization 1400, the detailed cluster view visualization 1500 provides several columns with detailed information for each placement request, including the prioritization score, the patient, the request time, the source of the request, the specialty type of the medical service associated with the request, the current location of the patient, the order priority, the level of care, and the event type. The detailed cluster view visualization 1500 also separates/distinguishes between the respective requests within the PEDS unit by requested bed type or level of care, which can include ACCUTE and ICU in this example implementation.

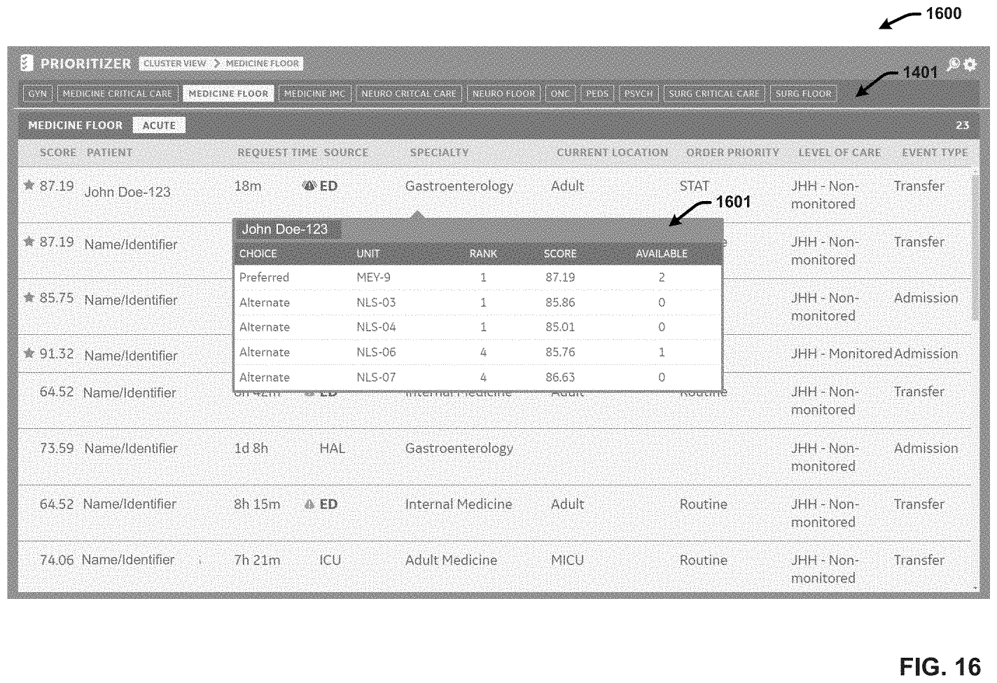

FIG. 16 presents an example GUI including another detailed cluster view visualization 1600 that provides a detailed view of the bed requests for acute beds types in the medicine floor service line, in accordance with one or more embodiments described herein. FIG. 16 further depicts a pop-up display window 1601 presented as an overlay on the detailed cluster view visualization 1600. In various embodiments, the specific requests and/or one or more specific table entries within specific columns can be selected to drill down on more detailed information respectively associated therewith. This additional information can be rendered in a pop-up display window, such as pop-up display window 1601. For example, in the embodiment shown, the data entries identifying the specialty of a particular request, (located in the “Specialty” column), can be selected to view additional information regarding patient prioritization details for the associated request in a pop-up display window. For instance, in the embodiment shown, the pop-up display window 1601 can be generated in response to selection of the “Gastroenterology” data entry in the specialty data filed for the placement request for patient John Doe—123. (In other implementations, any portion of the row associated with a particular request can be selected to view additional details associated with the request in the form of a pop-up display window overlay). The additional information included in a pop-up display window generated in response to selection of a request or selection of the specialty field data entry associated with a request can include information regarding the units where the request can be fulfilled and a priority of the request in the respective units. For example, as shown in the pop-up display window 1601, there are five different units within the gastroenterology where the patient can be placed. The pop-up display window 1601 further provides information indicating which of the five units is the preferred unit, the patient's rank relative to other patients for being placed in the respective units, the prioritization score associated with the request relative to the respective units, and the number of available units in the respective units. For example, in the embodiment shown, the patient is ranked number one in the preferred unit and the preferred unit has two available beds. Accordingly, a bed manager can quickly interact with visualization 1600 and 1601 to determine that the patient John Doe—123 should be assigned to one of the available beds in the preferred unit.

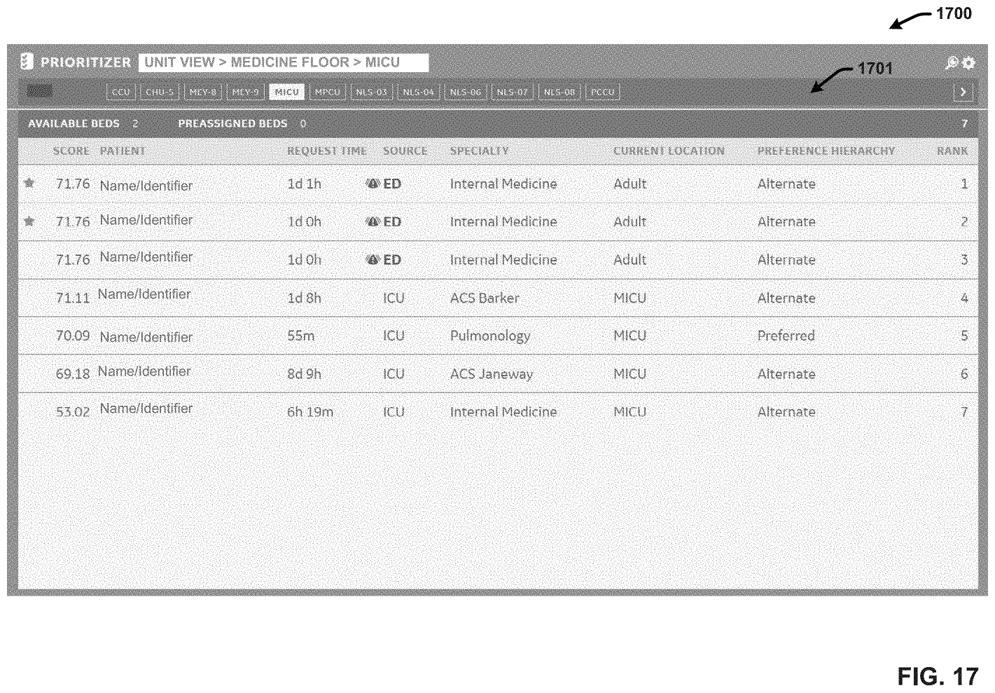

FIG. 17 presents an example GUI including a unit view visualization 1700 in accordance with one or more embodiments described herein. The unit view visualization can be used to be evaluate patient placement requests for a specific medical service line within a specific medical unit where the respective requests can be fulfilled. In this regard, the unit level view can help bed managers to visualize the competing demand in a selected unit. For example, in the embodiment shown, the unit view visualization 1700 presents patient placement requests in the medicine floor service line that can be placed in the MICU unit. A unit toolbar 1701 is provided that identifies and enables efficient selection of the different medical units within the selected service line to switch between viewing. For example, in the embodiment shown, the medicine floor service line includes twelve different medical units.

Similar to the detailed cluster view visualizations, the unit view visualization 1700 can present information in a table format that identifies patient placement requests, their respective prioritization scores, the patient associated with the request, the request time, the source type, the specialty, and the current location of the patient. The unit view visualization 1700 can also include information indicated whether the selected medical unit is a preferred or alternate unit for each request, and the ranking of each request within the selected unit. For example, in the embodiment shown, there are seven pending requests for the selected MICU unit, presented in ascending order from the top ranked (number 1) to the lowest ranked (number 7). The unit view visualization 1700 can also include information identifying the number of available beds and the number of pre-assigned beds in the selected unit. In this regard, the unit provides bed managers with an efficient way to identify the potential number of patients that can be placed in the selected unit and where this unit is in the preference tier hierarchy for each request.

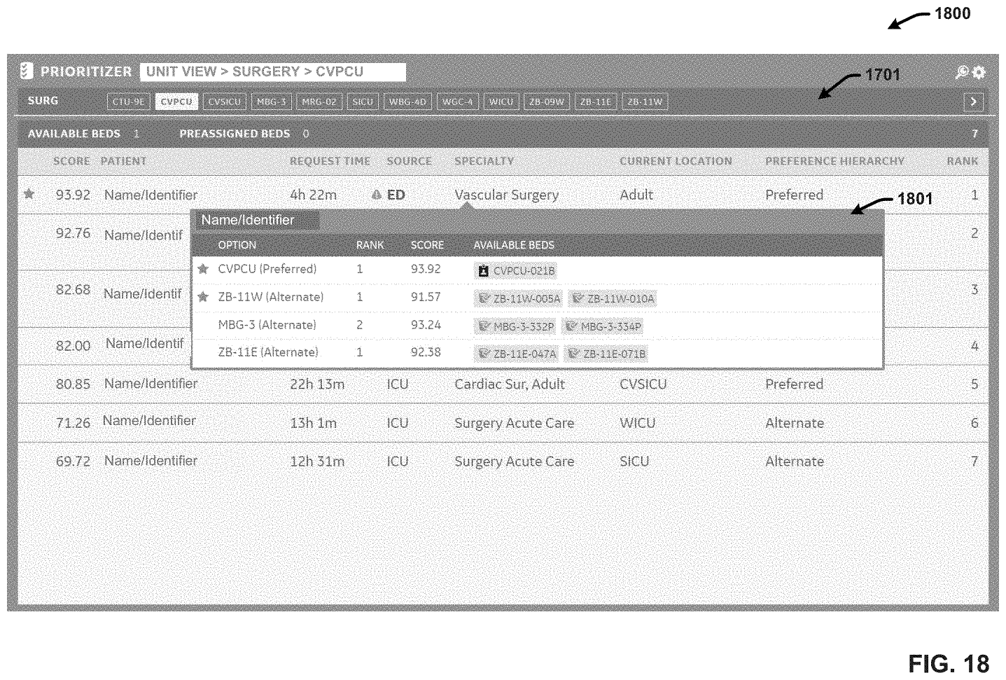

FIG. 18 presents an example GUI including another unit view visualization 1800 with a pop-up display window 1801, in accordance with one or more embodiments described herein. In the embodiment shown, the unit view is provided for the CVPCU unit within the surgery service line. Similar to the detailed cluster view visualizations, a specific request and/or one or more specific data entries associated with the request can be select to view additional details associated therewith in the form of a pop-up display window. For example, in the embodiment shown, the specialty data entry associated with a particular request can be selected to view additional information associated with the request. (In other implementations, any portion of the row associated with a particular request can be selected to view additional details associated with the request in the form of a pop-up display window overlay).

The pop-up display window 1801 can provide detailed information regarding the medical unit options associated with the selected request in addition to the currently selected medical unit. For example, in the embodiment shown, in addition to the CVPCU unit, three additional units included in the surgery service line are identified as medical unit options of the selected request. The information in pop-up display window 1801 indicates which unit is the preferred unit and which units are alternate units, the rank of the request in each unit, and the prioritization score associated with the request with respect to each unit. The information in the pop-up display window 1801 also includes information identifying the available beds in each unit and their associated EVS status. This feature is intended to help bed managers manage patient flow in congested sources by maximizing the number patients being placed from these sources. The aggregate count of the number of available beds and preassigned beds is also displayed.

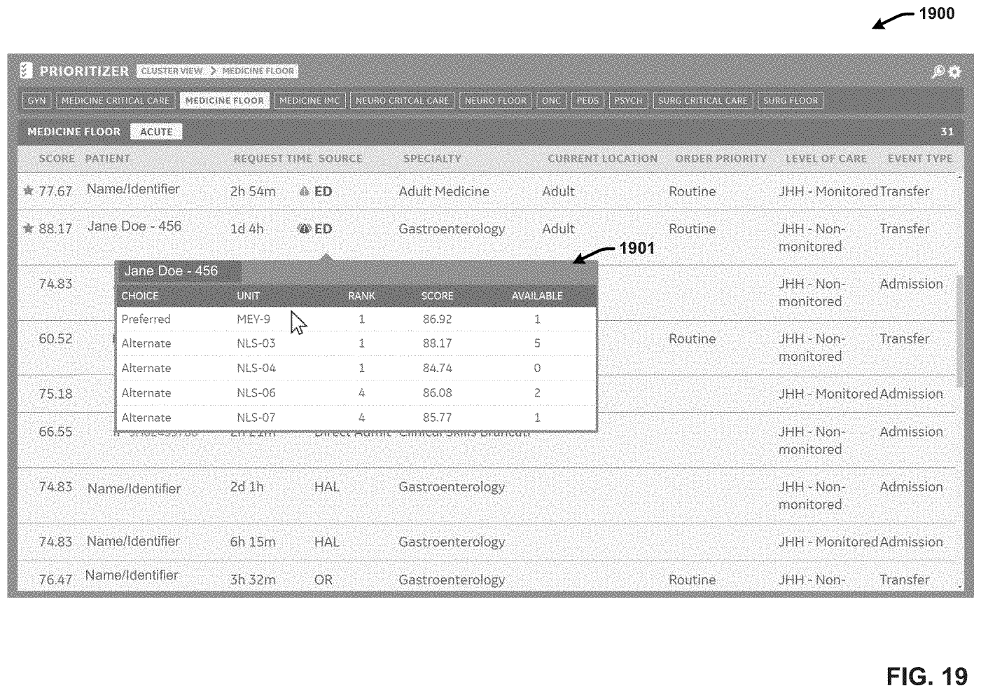

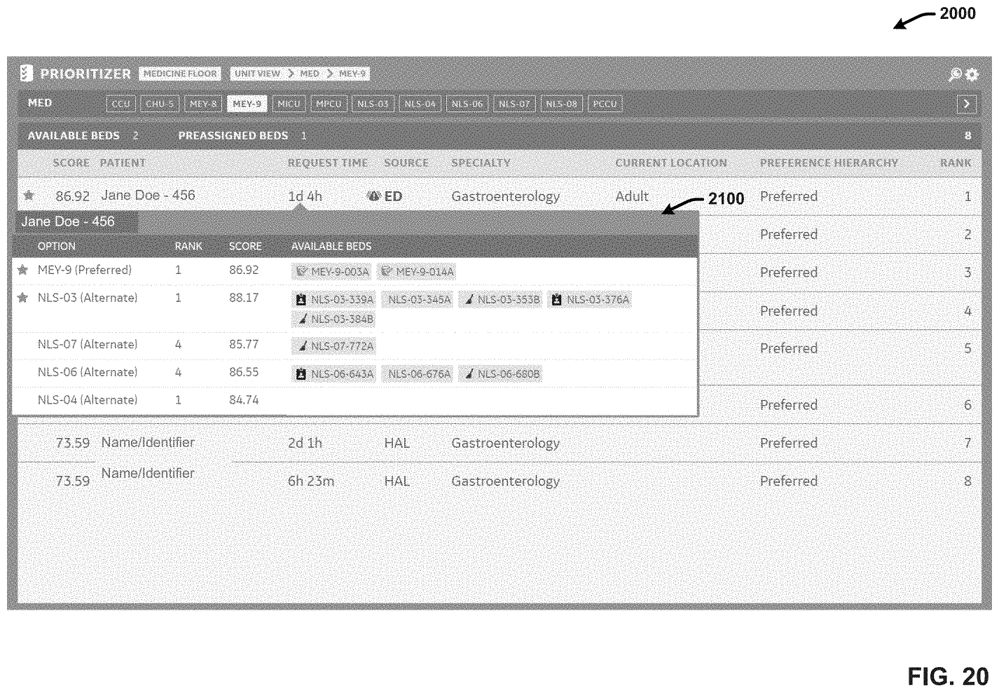

FIGS. 19 and 20 respectively present another detailed cluster view visualization 1900 and another detailed unit view visualization 2000 that can be presented to a bed manager in association an example use-case of the subject patient placement prioritization application 1202. The following example use-case demonstrates the manner in which a bed manager can use the subject patient placement prioritization application 1202 to efficiently determine how to assign patients to beds at a medical facility in real-time to optimize patient flow and the quality of care delivered to all patients of the medical facility. Repetitive description of like elements employed in respective embodiments is omitted for sake of brevity.

With reference to FIG. 19, consider a patient in ED (Jane Doe—456) managed by the Gastroenterology hospital service requesting a medicine floor bed as shown in the detailed cluster view visualization 1900. This patient has been in a ED bay, has been admitted to the hospital and also has an open bed request. The wait time of the bed request for this patient is approximately 28 hours. There is a patient flow alert for this patient displayed on the command center ED tile (e.g., indicated by the triangular symbol to the immediate left of the ED source entry). As can be seen in the pop-up display window 1901, MEY-9 (Meyer 9) is the preferred unit for this patient with several others as alternate units. This patient has been prioritized for placement (e.g., by the recommendation component 1104) as indicated by the star symbol associated with the patient's prioritization score of 88.17. In accordance with this example use-case, selection of (e.g., via clicking on) the MEY-9 unit row within the pop-up display window 1901 can result in the generation and presentation of the detailed unit view visualization 2000 shown in FIG. 20.

With reference to FIG. 20, the unit view visualization 2000 indicates that there are two available beds within the MEY-9 unit one of the available bed has already been preassigned. There are 8 patients (e.g., ranked 1-8) waiting to get into the MEY-9 unit who are managed by the same hospital service (e.g., the medicine floor). The MEY-9 unit is the preferred unit for all 8 patients. The prioritization score (86.92) for patient W.GIA's bed request within the MEY-9 unit is the highest relative to other competing requests and hence ranked first. The pop-up display window 2100 further identifies the other unit options where this request can be fulfilled, the request rank relative to the other unit options, associated prioritization scores, and the available beds in the other alternate units. As shown in the pop-up display window 2100, this placement request is ranked first in three units, including MEY-9, NLS-03 and NLS-04. A star symbol is provided for both MEY-9 and NLS-03 to indicate that there is at least one available bed in these units where the request is prioritized for placement. Based on evaluation of the information associated with the request in the detailed cluster view visualization 1900 and the unit view visualization 2000, a bed manger can decide that this patient should be placed now to an available bed in either the MEY-9 unit or the NLS-03 unit. Because the MEY-9 is the preferred unit, this is the optimal choice for placement.

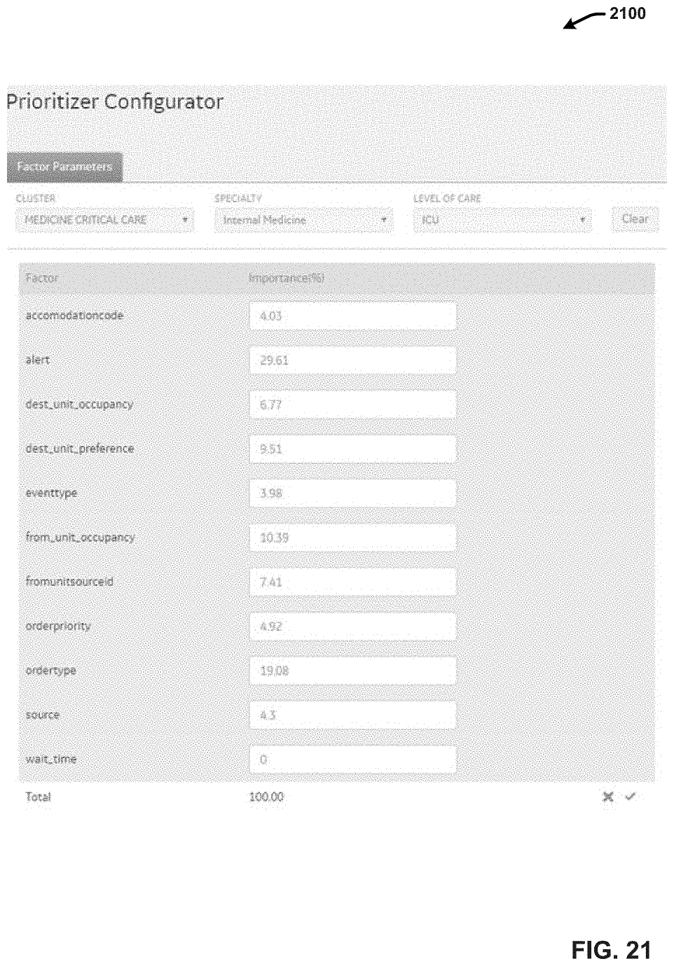

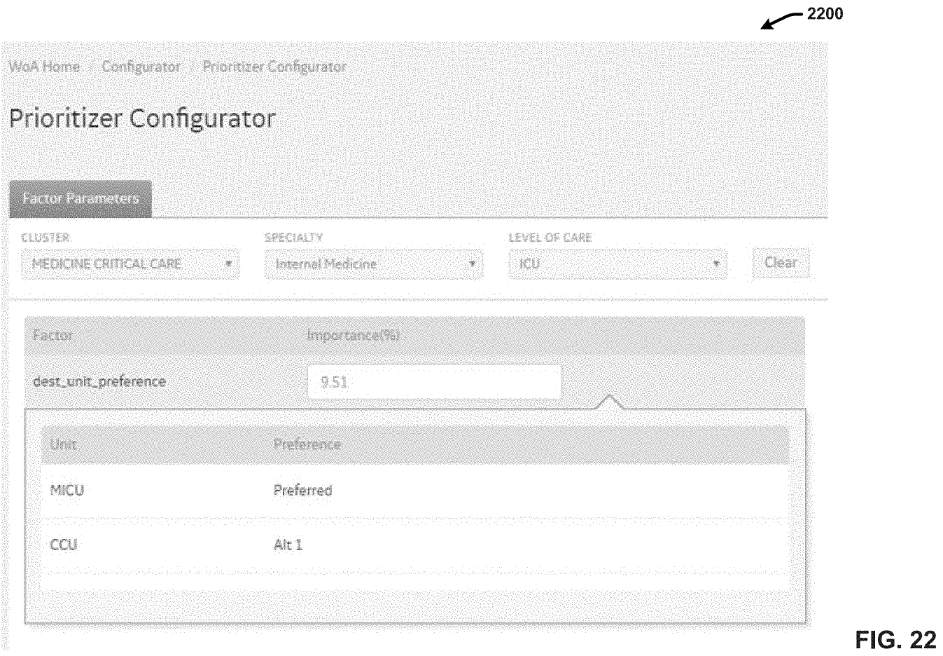

FIGS. 21 and 22 present example GUIs that facilitate use-based configuration of prioritization model parameters in accordance with one or more embodiments described herein. FIG. 21 presents a configuration GUI 2100 that allows the bed managers to adjust the weighting of different factors included in a prioritization model to reflect their operational policies. FIG. 22 presents another configuration GUI 2200 that allows the bed managers to change their preference hierarchy to optimize care for patients. In one or more embodiments, the configuration features and functionalities described with reference to GUIs 2100 and 2200 can be facilitated by the configuration component 1208. Repetitive description of like elements employed in respective embodiments is omitted for sake of brevity.

With reference to FIGS. 21 and 22, the configuration GUI 2100 and 2200 allow bed managers to manage the weights of the prioritization model at the cluster level or the hospital service-bed type level, for easy editing of the parameters of the model. Through these configuration GUIs, a bed manager can change weights, factor values and preference hierarchy. The weights of the model as well as the normalized factor values are also editable. In some implementations, the configuration component 1208 can create snapshots of the parameters and roll back the parameter values to default or the previous snapshot stored values. These GUIs enable the bed managers to synchronize the placement prioritization model with the operational priorities and policies of bed management of a particular medical facility that the system is applied to. Simple constraint checks (such as sum of weights should add up to 100) can be provided to avoid data entry errors and prevent violating the assumptions of the weighted sum model.

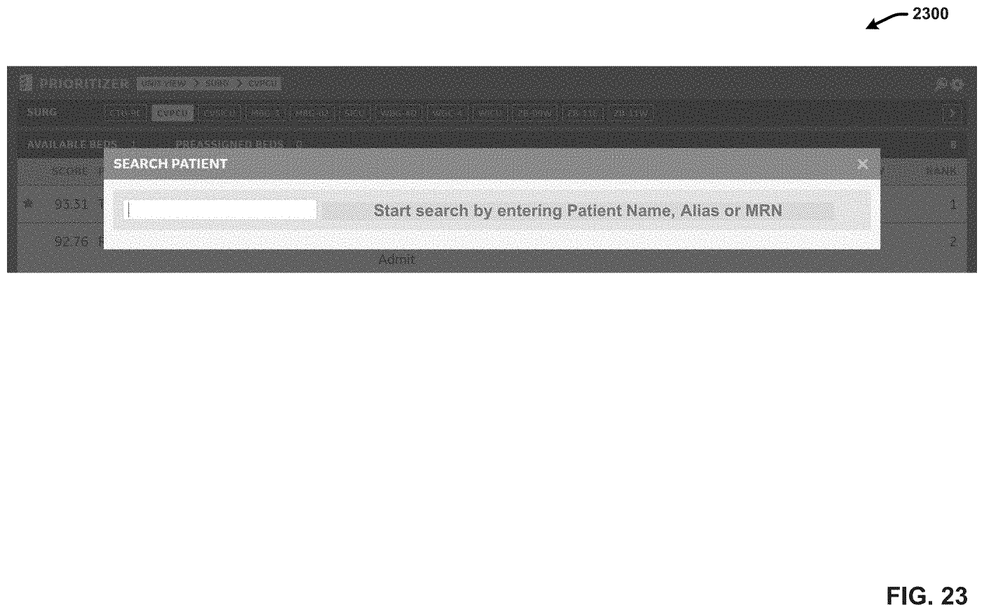

FIG. 23 presents an example search GUI 2300 that can be used to find specific patient placement requests in accordance with one or more embodiments described herein. In the embodiment shown, the search GUI 2300 can allow users to search for specific patient placement requests by one or more criteria, including patient medical record number (MRN), patient name or alias. In response to entry of one or more of the defined criteria, the query component 1206 can be configured to identify the corresponding placement request for the patient, and present information (e.g., in a detailed cluster view) identifying the request, the prioritization score for the request relative to other placement requests within the same medical service/bed type combination, and other details associated with the request described herein (e.g., presented in the cluster view).

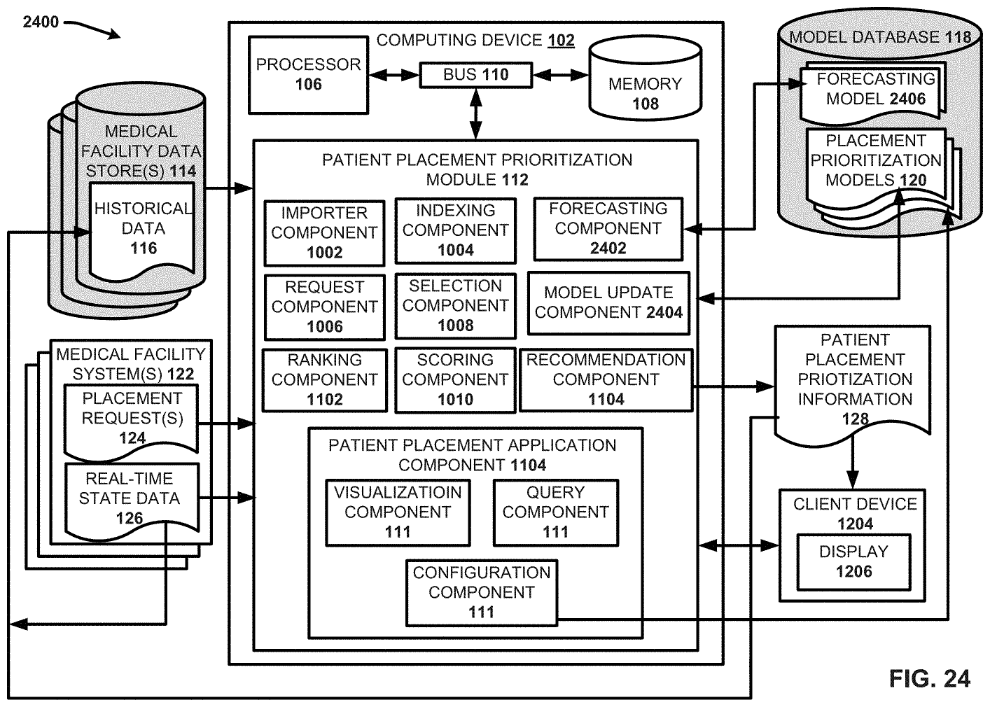

FIG. 24 illustrates a block diagram of an example, non-limiting system 2400 that provides for employing a multifactorial, machine-learning based prioritization framework for optimizing patient placement in real-time, in accordance with one or more embodiments of the disclosed subject matter. System 2400 can include same or similar features and functionalities as system 1200 with the addition of forecasting component 2402, model update component 2404, one or more forecasting models 2406, and the medical facility data stores including the historical data 116. Repetitive description of like elements employed in respective embodiments is omitted for sake of brevity.

The forecasting component 2402 can be configured to employ one or more machine learning techniques to facilitate inferring information regarding forecasted contextual states of the healthcare facility and/or forecasted events associated with the healthcare facility that system 2400 is applied to in association with facilitating prioritization patient placements to achieve optimal patient flow and quality of care. For example, the forecasting component 2404 can infer information regarding forecasted bed availability, forecasted occupancy levels, and the like. In another example, the forecasting component 2402 can infer event information regarding probable delays in patient workflows, probable complications to patient procedures, probable patient needs, predicted LOS, and the like. In this regard, the forecasting component 2402 can be configured to identify and learn patterns in the historical data 116 (using one or more machine learning techniques), to make inferences regarding forecasted contextual states of the healthcare facility and/or forecasted events associated with the healthcare facility. In accordance with these embodiments, the historical data 116 can include not only the historical operations and performance data regarding historical patient placement patterns, but also collated patient placement prioritization information 128 generated over the course of operation of the healthcare facility and collated real-time state data 126 regarding various contextual states including bed availability, occupancy levels, supply distribution and availability, clinician location and activity information, scheduling information and the like.

For example, in one or more embodiments, the forecasting component 2402 can be configured to evaluate patterns in historical bed usage and EVS status to determine or infer (using artificial intelligence and one or more classifiers) correlations between various parameters reflected in the historical data 116 and information regarding when certain beds will become available (e.g., a predicted point in time, a duration of time or waiting period). For example, the forecasting component 2402 can identify correlations between types of beds, medical units where the beds are located, types of patient requests assigned to different beds, characteristics of the requests, timing of initial assignment of requests to beds (e.g., time of day/day of week), EVS status, duration of time beds are associated with respective EVS status, and the like, with timing and duration of bed occupancy and bed availability.

The forecasting component 2402 can also learn patterns in occupancy levels of the healthcare facility as whole as well as respective medical units and bed types, to infer information regarding predicted occupancy patterns. In this regard, the predicted occupancy information can include a predicted amount and distribution of patients as a function of time/date, predicted number and distribution of incoming patients, type of medical needs and associated bed request for the predicted incoming patients, and the like. The predicted occupancy information can account for correlations in historical occupancy levels and time (time of day and day of week), correlations in types of requests received at certain times of day and day of week, and the like. In some embodiments, the forecasting component 2402 can also learn patterns in patient workflows and associated complications and delays to predict information regarding durations of time a patient will remain in a particular bed, predicted bed transfer requests, predicted LOS and the like. The forecasting component 2402 can also predict information regarding predicted patient needs and associated bed transfer requests based on patterns in patient workflow information. For example, the forecasting component 2402 can predict that based on the number of patients admitted for condition or procedure X in the last 8 hours, there will be Y amount of transfer requests for bed type N and medical unit M in the next 24 hours.

In one or more embodiments, based on this machine learning analysis of the historical data 116, using artificial intelligence and one or more classifiers, the forecasting component 2402 can be configured to develop one or more forecasting models 2406 that can respectively be configured to generate information regarding predicted bed availability, occupancy levels, patient workflow delays and needs, predicted transfer request types and distribution, LOS, and based on real-time state data 126 received over the course of operation of the healthcare facility, the current time of day and day of year, current monitored patient workflow data and the like. For example, the forecasting component 2402 can be configured to generate a bed availability forecasting model (e.g., one or the forecasting models 2406) that can be configured to generate forecasted bed availability information based on learned parameter correlations in the historical data 116. These models can be stored in model database 118 (e.g., as forecasting models 2406) and used by the forecasting component 2402 to generate output information regarding these forecasted healthcare facility event and conditional data points in real-time these probable data points in real time.

In some embodiments, the forecasted information regarding one or more contextual states or events associated with the medical facility determined by the forecasting component 2402 (e.g., using the forecasting models 2406) can be accounted for in the prioritization scores determined for respective placement requests by the scoring component 1010 using the placement prioritization models 120. With these embodiments, the model update component 2404 can be configured to update the placement prioritization models 120 to account for forecasted information regarding bed availability, occupancy levels, patient workflow delays and needs, predicted transfer request types and distribution, LOS, and the like. For example, with respect to forecasted bed availability information, the model update component 2404 can be configured to update the placement prioritization models 120 to be configured to generate prioritization scores for patient placement requests that further account for the predicted timing of availability of beds at which the respected requests can be fulfilled. In accordance with this example, a prioritization score for a specific type of placement request (e.g., having a distinct bed type and medical service combination) with respect to a specific medical unit generated using the updated placement prioritization model can further account for the predicted duration of times until respective unavailable or occupied bed in the medical unit will become available. The model update component 2404 can similarly update the respective placement prioritization models 120 to determine prioritization scores as function of one or more other predicted events/conditions, including occupancy levels, patient workflow delays and needs, predicted transfer request types and distribution, LOS, and the like.

In another embodiment, the recommendation component 1104 can be configured to use forecasted information regarding predicted contextual states and/or predicted events associated with operation of the medical facility, along with priority scores determined for pending placement requests, to further optimize recommendations regarding how to prioritize patient placements. For example, one of the critical questions that bed managers are challenged to answer is whether to wait for a bed in the preferred unit or assign a patient to a less preferred medical unit that currently has an available bed. It is very difficult to evaluate this trade-off without a structured systematic framework. To help address this question, the recommendation component 1104 can combine forecasts of bed availability along with historical placement patterns for similar requests, hospital states and other forecasted information to further determine which requests to recommend for placement to available beds in respective medical units and when. In this regard, in some implementations, the recommendation component 1104 can further determine a recommendation regarding whether to assign a patient to an available bed in a medical unit based on the prioritization score associated with the requests, the number of available beds in the preferred unit, the number of alternate units, the number of available beds in the alternate units, and predicted bed availability in the preferred unit and the alternate units. The recommendation component 1104 can also determine or infer recommendations regarding where and when to assign patients to beds based on their respective request prioritization scores and predicted occupancy levels, predicted patient workflow delays and needs, predicted transfer request types and distribution, predicted LOS and the like. For example, if a medical unit X has relatively low current occupancy levels, and the medical facility is likely to receive a surge in requests for medical unit Y, which currently has high occupancy levels, the recommendation component can be configured recommend pre-assigning request to medical unit X and/or recommend moving patients from medical unit Y to medical unit X to prepare for the upcoming events. In another example, a particular patient request is likely to occupy a potential bed for duration of time over N hours and several other patients with workflow needs with relatively short predicted occupancy times are waiting for the same bed, the recommendation component 1104 can recommend placing the particular patient request to an alternative bed to optimize patient flow.

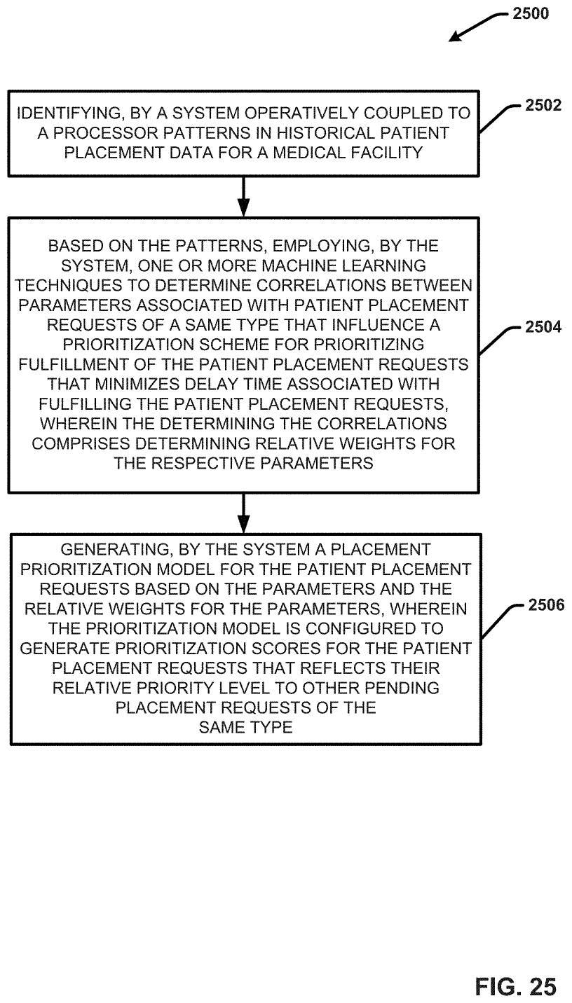

FIGS. 25-27 illustrate flow diagrams of example, non-limiting methods that employ a multifactorial, machine-learning based prioritization framework to facilitate optimizing patient placement to beds at a medical facility. While, for purposes of simplicity of explanation, the methodologies are shown and described as a series of acts, the disclosed subject matter is not limited by the order of acts, as some acts can occur in different orders and/or concurrently with other acts from that shown and described herein. For example, those skilled in the art will understand and appreciate that a methodology can alternatively be represented as a series of interrelated statuses or events, such as in a state diagram. Moreover, not all illustrated acts may be required to implement a methodology in accordance with the disclosed subject matter. Additionally, it is to be appreciated that the methodologies disclosed in this disclosure are capable of being stored on an article of manufacture to facilitate transporting and transferring such methodologies to computers or other computing devices. The following methods facilitate enhanced assessing risk associated with firewall rules.

Referring now to FIG. 25 presented an example, high level flow diagram of a computer-implemented process 2500 for employing a multifactorial, machine-learning based prioritization framework to facilitate optimizing patient placement, in accordance with one or more embodiments of the disclosed subject matter. Repetitive description of like elements employed in respective embodiments is omitted for sake of brevity.

At 2502, a system, operatively coupled to a processor, identifies patterns in historical patient placement data for a medical facility. At 2504, based on the patterns, the system employs one or more machine learning techniques to determine correlations between parameters associated with patient placement requests of a same type that influence a prioritization scheme for prioritizing fulfillment of the patient placement requests that minimizes delay time associated with fulfilling the patient placement requests, wherein the determining the correlations comprises determining relative weights for the respective parameters. At 2506, the system generates a placement prioritization model for the patient placement requests based on the parameters and the relative weights for the parameters, wherein the prioritization model is configured to generate prioritization scores for the patient placement requests that reflects their relative priority level to other pending placement requests of the same type.

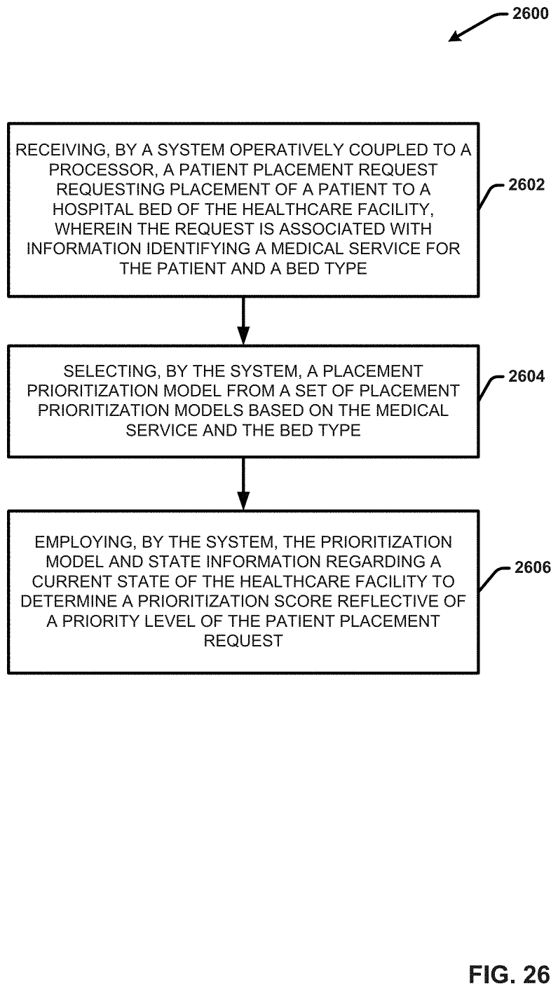

FIG. 26 illustrates another example, system flow diagram 2600 of another computer-implemented process for employing a multifactorial, machine-learning based prioritization framework to facilitate optimizing patient placement, in accordance with one or more embodiments of the disclosed subject matter. Repetitive description of like elements employed in respective embodiments is omitted for sake of brevity.

At 2602, a system operatively coupled to a processor, receives a patient placement request requesting placement of a patient to a hospital bed of the healthcare facility, wherein the request is associated with information identifying a medical service for the patient and a bed type. At 2604, the system selects a placement prioritization model from a set of placement prioritization models based on the medical service and the bed type. At 2606, the system employs the prioritization model and state information regarding a current state of the healthcare facility to determine a prioritization score reflective of a priority level of the patient placement request.

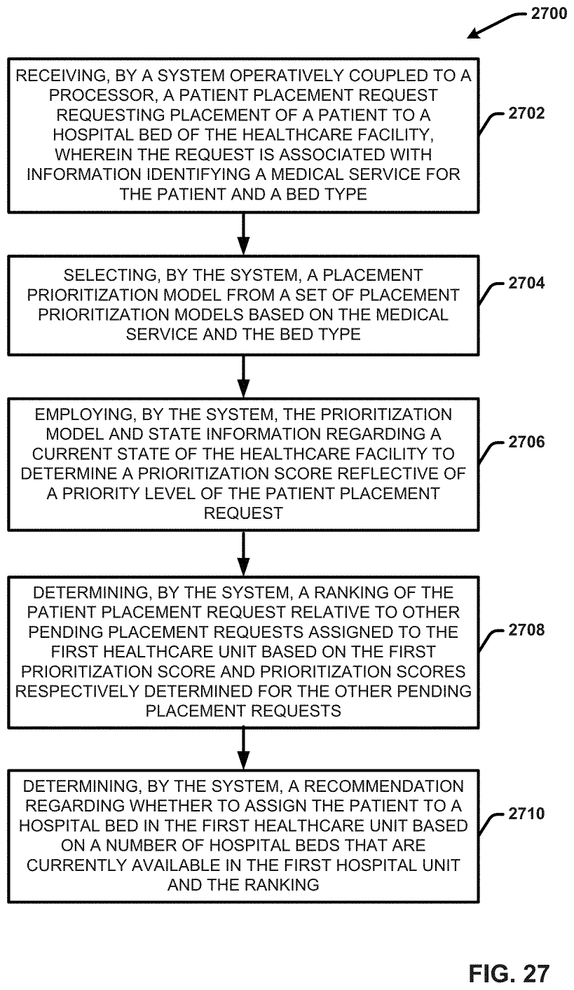

FIG. 27 illustrates another example, high level flow diagram of another computer-implemented process 2700 for employing a multifactorial, machine-learning based prioritization framework to facilitate optimizing patient placement, in accordance with one or more embodiments of the disclosed subject matter. Repetitive description of like elements employed in other embodiments described herein is omitted for sake of brevity.

At 2702, a system operatively coupled to a processor, receives a patient placement request requesting placement of a patient to a hospital bed of the healthcare facility, wherein the request is associated with information identifying a medical service for the patient and a bed type. At 2704, the system selects a placement prioritization model from a set of placement prioritization models based on the medical service and the bed type. At 2706, the system employs the prioritization model and state information regarding a current state of the healthcare facility to determine a prioritization score reflective of a priority level of the patient placement request. At 2708, the system determines a ranking of the patient placement request relative to other pending placement requests assigned to the first healthcare unit based on the first prioritization score and prioritization scores respectively determined for the other pending placement requests. At 2710, the system determines a recommendation regarding whether to assign the patient to a hospital bed in the first healthcare unit based on a number of hospital beds that are currently available in the first hospital unit and the ranking.

The disclosed systems, methods and computer readable media provide a centralized, explicit and transparent framework that facilitates prioritizing the placement of patients in an inpatient medical facility. This framework is data driven and quantitatively evaluates patient placement options based on multiple different factors and bed availability within the system. The framework is further robust, transparent and adaptable to almost any hospital with varied policies and rules of operations the disclosed techniques can combine a large number of quantitative and qualitative data from disparate data sources and employ multicriteria decision making on a multitude of different factors (e.g. varying patient needs and preferences, different rules for allocating beds within the same unit). The disclosed techniques thereby account for data at the granular level and provide organizational alignment by incorporating the operating rules of the medical facility.

In particular, the disclosed techniques integrate multicriteria decision (MCD) making with supervised machine learning techniques to deduce bed manager preferences and optimal placement prioritization schemes from historical data. In some embodiments, the patient placement prioritization problem can be modeled using a multi-criteria weighted sum model with an additive utility function. Estimation of weights in the weighted sum model can be performed using machine learning techniques rather than the conventional techniques. For example, a regression tree methodology can be used to estimate the weights of the additive utility function in the multicriteria weighted sum model. The regression tree methodology outputs variable importance scores that can be used to estimate the weights of the additive utility function. Several different weighted sum models can be tailored to different types of patient placement requests and/or medical units depending on the operational policy of the hospital. The parameters of these models along with unfulfilled bed requests are input into a scoring model and are used to estimate a prioritization score in real-time. This information is further combined with availability of beds in feasible units to prioritize placements. This method is computationally very efficient and can operate in real-time.

The various systems, methods and computer readable media described herein further provide substantial technical improvements in medical field with respect to facilitating bed management in inpatient medical facilities. For example, even with the state of the art advances in technology, most of the required information needed to make informed bed management decisions (e.g., bed availability, EVS status, bed requests, occupancy levels, forecasts, etc.) resides in different systems, applications, screens or databases. The disclosed systems, methods and computer readable media however combine the information pertinent to prioritizing placements using in single application so that decision makers can quickly assess the situation, evaluated various placement options, and make informed decisions regarding prioritization patient placements. The application can provide comprehensive information about pending placement requests and the available placement options, and enable bed managers to view and control the patient flow from the moment patients enter the hospital. In this regard, the application can provide a centralized bed management function that allows real-time understanding of bed supply and demand across the entire hospital enterprise. Since this system is centralized, bed managers have a holistic view of the system and can make better decisions. Further during high volume periods, the stress on the bed managers are immense. Presentation makes a huge different in how the information gets used. The disclosed bed prioritization application however breaks complexities into manageable components brought together in a systematic GUI that can be easily navigated. The application also includes a configuration interface that allows bed managers to make updates to the prioritization models.

One or more embodiments can be a system, a method, and/or a computer program product at any possible technical detail level of integration. The computer program product can include a computer readable storage medium (or media) having computer readable program instructions thereon for causing a processor to carry out one or more aspects of the present embodiments.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the entity's computer, partly on the entity's computer, as a stand-alone software package, partly on the entity's computer and partly on a remote computer or entirely on the remote computer or server. In the latter scenario, the remote computer can be connected to the entity's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It can be understood that each block of the flowchart illustrations and/or block diagrams, and combinations of blocks in the flowchart illustrations and/or block diagrams, can be implemented by computer readable program instructions.

These computer readable program instructions can be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession may, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

In connection with FIG. 28, the systems and processes described below can be embodied within hardware, such as a single integrated circuit (IC) chip, multiple ICs, an application specific integrated circuit (ASIC), or the like. Further, the order in which some or all of the process blocks appear in each process should not be deemed limiting. Rather, it should be understood that some of the process blocks can be executed in a variety of orders, not all of which can be explicitly illustrated herein.

With reference to FIG. 28, an example environment 2800 for implementing various aspects of the claimed subject matter includes a computer 2802. The computer 2802 includes a processing unit 2804, a system memory 2806, a codec 2835, and a system bus 2808. The system bus 2808 couples system components including, but not limited to, the system memory 2806 to the processing unit 2804. The processing unit 2804 can be any of various available processors. Dual microprocessors and other multiprocessor architectures also can be employed as the processing unit 2804.

The system bus 2808 can be any of several types of bus structure(s) including the memory bus or memory controller, a peripheral bus or external bus, or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 13284), and Small Computer Systems Interface (SCSI).

The system memory 2806 includes volatile memory 2810 and non-volatile memory 2812, which can employ one or more of the disclosed memory architectures, in various embodiments. The basic input/output system (BIOS), containing the basic routines to transfer information between elements within the computer 2802, such as during start-up, is stored in non-volatile memory 2812. In addition, according to present innovations, codec 2835 can include at least one of an encoder or decoder, wherein the at least one of an encoder or decoder can consist of hardware, software, or a combination of hardware and software. Although, codec 2835 is depicted as a separate component, codec 2835 can be contained within non-volatile memory 2812. By way of illustration, and not limitation, non-volatile memory 2812 can include read only memory (ROM), programmable ROM (PROM), electrically programmable ROM (EPROM), electrically erasable programmable ROM (EEPROM), Flash memory, 3D Flash memory, or resistive memory such as resistive random access memory (RRAM). Non-volatile memory 2812 can employ one or more of the disclosed memory devices, in at least some embodiments. Moreover, non-volatile memory 2812 can be computer memory (e.g., physically integrated with computer 2802 or a mainboard thereof), or removable memory. Examples of suitable removable memory with which disclosed embodiments can be implemented can include a secure digital (SD) card, a compact Flash (CF) card, a universal serial bus (USB) memory stick, or the like. Volatile memory 2810 includes random access memory (RAM), which acts as external cache memory, and can also employ one or more disclosed memory devices in various embodiments. By way of illustration and not limitation, RAM is available in many forms such as static RAM (SRAM), dynamic RAM (DRAM), synchronous DRAM (SDRAM), double data rate SDRAM (DDR SDRAM), and enhanced SDRAM (ESDRAM) and so forth.

Computer 2802 can also include removable/non-removable, volatile/non-volatile computer storage medium. FIG. 28 illustrates, for example, disk storage 2814. Disk storage 2814 includes, but is not limited to, devices like a magnetic disk drive, solid state disk (SSD), flash memory card, or memory stick. In addition, disk storage 2814 can include storage medium separately or in combination with other storage medium including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage 2814 to the system bus 2808, a removable or non-removable interface is typically used, such as interface 2816. It is appreciated that disk storage 2814 can store information related to an entity. Such information might be stored at or provided to a server or to an application running on an entity device. In one embodiment, the entity can be notified (e.g., by way of output device(s) 2836) of the types of information that are stored to disk storage 814 or transmitted to the server or application. The entity can be provided the opportunity to opt-in or opt-out of having such information collected or shared with the server or application (e.g., by way of input from input device(s) 2828).

It is to be appreciated that FIG. 28 describes software that acts as an intermediary between entities and the basic computer resources described in the suitable operating environment 2800. Such software includes an operating system 2818. Operating system 2818, which can be stored on disk storage 2814, acts to control and allocate resources of the computer system 2802. Applications 2820 take advantage of the management of resources by operating system 2818 through program modules 2824, and program data 2826, such as the boot/shutdown transaction table and the like, stored either in system memory 2806 or on disk storage 2814. It is to be appreciated that the claimed subject matter can be implemented with various operating systems or combinations of operating systems.

An entity enters commands or information into the computer 2802 through input device(s) 2828. Input devices 2828 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 2804 through the system bus 2808 via interface port(s) 2830. Interface port(s) 2830 include, for example, a serial port, a parallel port, a game port, and a universal serial bus (USB). Output device(s) 2836 use some of the same type of ports as input device(s) 2828. Thus, for example, a USB port can be used to provide input to computer 2802 and to output information from computer 2802 to an output device 2836. Output adapter 2834 is provided to illustrate that there are some output devices 2836 like monitors, speakers, and printers, among other output devices 2836, which require special adapters. The output adapters 2834 include, by way of illustration and not limitation, video and sound cards that provide a means of connection between the output device 2836 and the system bus 2808. It should be noted that other devices or systems of devices provide both input and output capabilities such as remote computer(s) 2838.

Computer 2802 can operate in a networked environment using logical connections to one or more remote computers, such as remote computer(s) 2838. The remote computer(s) 2838 can be a personal computer, a server, a router, a network PC, a workstation, a microprocessor based appliance, a peer device, a smart phone, a tablet, or other network node, and typically includes many of the elements described relative to computer 2802. For purposes of brevity, only a memory storage device 2840 is illustrated with remote computer(s) 2838. Remote computer(s) 2838 is logically connected to computer 2802 through a network interface 2842 and then connected via communication connection(s) 2844. Network interface 2842 encompasses wire or wireless communication networks such as local-area networks (LAN) and wide-area networks (WAN) and cellular networks. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection(s) 2844 refers to the hardware/software employed to connect the network interface 2842 to the system bus 2808. While communication connection 2844 is shown for illustrative clarity inside computer 2802, it can also be external to computer 2802. The hardware/software necessary for connection to the network interface 2842 includes, for exemplary purposes only, internal and external technologies such as, modems including regular telephone grade modems, cable modems and DSL modems, ISDN adapters, and wired and wireless Ethernet cards, hubs, and routers.

While the subject matter has been described above in the general context of computer-executable instructions of a computer program product that runs on a computer and/or computers, those skilled in the art will recognize that this disclosure also can or can be implemented in combination with other program modules. Generally, program modules include routines, programs, components, data structures, etc. that perform particular tasks and/or implement particular abstract data types. Moreover, those skilled in the art will appreciate that the inventive computer-implemented methods can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments where tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” and/or “exemplary” are utilized to mean serving as an example, instance, or illustration and are intended to be non-limiting. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of entity equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim. The descriptions of the various embodiments have been presented for purposes of illustration, but are not intended to be exhaustive or limited to the embodiments disclosed. Many modifications and variations can be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system for prioritizing patient placements at a healthcare facility, comprising:
a memory that stores computer executable components; and
a processor that executes the computer executable components stored in the memory, wherein the computer executable components comprise:
a request component that receives a patient placement request requesting placement of a patient to a hospital bed of the healthcare facility, wherein the request is associated with care requirement information identifying care requirements for the patient, including a medical service for the patient and a bed type;
a selection component that selects a placement prioritization model from a set of placement prioritization models based on the medical service and the bed type; and
a scoring component that employs the prioritization model and state information regarding a current state of the healthcare facility to determine a prioritization score reflective of a priority level of the patient placement request.

2. The system of claim 1, wherein the computer executable components further comprise:
a model generation component that employs one or more machine learning techniques to learn placement patterns of bed managers from historical data located at disparate network accessible data sources, and determines key parameters and medical unit preferences that influence prioritization of patient placement requests.

3. The system of claim 2, wherein the model generation component further generates respective placement prioritization models of the set of placement prioritization models based on the placement patterns, the key parameters and the medical unit preferences, and wherein the model generation component generates a different placement prioritization model for different combinations of medical services and bed types.

4. The system of claim 1, wherein the healthcare facility comprises one or more hospital beds at which the patient can be placed based on the care requirements for the patient, and wherein the computer executable components further comprise:
a recommendation component that provides a recommendation regarding whether to wait to place the patient or place the patient in a bed of the one or more hospital beds.

5. The system of claim 1, wherein the patient placement models respectively comprise weighted sum models developed using machine learning analysis of historical operations data and historical patient placement data for the healthcare facility.

6. The system of claim 1, wherein the healthcare facility comprises different medical units, wherein the medical service and the bed type are associated with a first medical unit of the different medical units, and wherein the prioritization score is a first prioritization score that reflects a first priority level of the patient placement request within the first medical unit.

7. The system of claim 6, wherein the computer executable components further comprise:
a ranking component that determines a ranking of the patient placement request relative to other pending placement requests assigned to the first medical unit based on the first prioritization score and prioritization scores respectively determined for the other pending placement requests.

8. The system of claim 7, wherein the computer executable components further comprise:
a recommendation component that determines a recommendation regarding whether to assign the patient to a bed in the first medical unit based on a number of beds that are currently available in the first medical unit and the ranking.

9. The system of claim 8, wherein the computer executable components further comprise:
a visualization component that generates a visualization that visually depicts the ranking and the recommendation for rendering via a display screen of a device.

10. The system of claim 6, wherein the medical service and the bed type are associated with a second medical unit of the different medical units and wherein the scoring component further employs the prioritization model and the state information to determine a second prioritization score reflective of a second priority level of the patient placement request within the second medical unit.

11. The system of claim 10, wherein the computer executable components further comprise:
a recommendation component that determines a recommendation regarding whether to assign the patient to a first hospital bed in the first medical unit or a second hospital bed in the second medical unit based on the first prioritization score, the second prioritization score, and a number of hospital beds that are currently available in the first medical unit and the second h unit.

12. The system of claim 11, wherein the computer executable components further comprise:
a bed availability forecasting component that employs artificial intelligence and one or more classifiers to forecast timing of bed availability within the first medical unit and the second medical unit based on the state information and historical patient workflow information, and wherein the recommendation component further determines the recommendation based on the timing of bed availability.

13. The system of claim 6, wherein the selection component further selects the placement prioritization model from the set of placement prioritization models based on the first medical unit.

14. The system of claim 13, wherein the medical service and the bed type are associated with a second medical unit of the different medical units, wherein the selection component further selects a second placement prioritization model from the set of placement prioritization models based on the second medical unit, the medical service and the bed type, and wherein the scoring component further employs the second placement prioritization model and the state information determine a second prioritization score reflective of a second priority level of the patient placement request within the second medical unit.

15. A method, comprising:
receiving, by a system operatively coupled to a processor, a patient placement request requesting placement of a patient to a hospital bed of a healthcare facility, wherein the request is associated with information identifying a medical service for the patient and a bed type;
selecting, by the system, a placement prioritization model from a set of placement prioritization models based on the medical service and the bed type; and
employing, by the system, the prioritization model and state information regarding a current state of the healthcare facility to determine a prioritization score reflective of a priority level of the patient placement request.

16. The method of claim 13, wherein the patient placement models respectively comprise weighted sum models developed using machine learning analysis of historical operations data and historical patient placement data for the healthcare facility.

17. The method of claim 13, wherein the healthcare facility comprises different medical units, wherein the medical service and the bed type are associated with a first medical unit of the different medical units, and wherein the prioritization score is a first prioritization score that reflects a first priority level of the patient placement request within the first medical unit.

18. The method of claim 17, further comprising:
determining, by the system, a ranking of the patient placement request relative to other pending placement requests assigned to the first medical unit based on the first prioritization score and prioritization scores respectively determined for the other pending placement requests.

19. The method of claim 18, further comprising:
determining, by the system, a recommendation regarding whether to assign the patient to a hospital bed in the first medical unit based on a number of hospital beds that are currently available in the first medical unit and the ranking.

20. The method of claim 19, further comprising:
generating, by the system, a visualization that visually depicts the ranking and the recommendation for rendering via a display screen of a device.

21. The method of claim 17, wherein the medical service and the bed type are associated with a second medical unit of the different medical units and wherein the method further comprises:
employing, by the system, the prioritization model and the state information to determine a second prioritization score reflective of a second priority level of the patient placement request within the second medical unit.

22. A machine-readable storage medium, comprising executable instructions that, when executed by a processor, facilitate performance of operations, comprising:
receiving a patient placement request requesting placement of a patient to a hospital bed of a healthcare facility, wherein the request is associated with information identifying a medical service for the patient and a bed type;
selecting a placement prioritization model from a set of placement prioritization models based on the medical service and the bed type; and
employing the prioritization model and state information regarding a current state of the healthcare facility to determine a prioritization score reflective of a priority level of the patient placement request.

23. The machine-readable storage medium of claim 22, wherein the patient placement models respectively comprise weighted sum models developed using machine learning analysis of historical operations data and historical patient placement data for the healthcare facility.
